SEQUENCE LISTING (13 sequences)

SEQ ID NO: 1 - protein, 470 aa, synthetic construct
  REGION 1..470: note = Heavy chain of anti-TROP2 antibody
MKHLWFFLLLVAAPRWVLSQVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 234 aa, synthetic construct
  REGION 1..234: note = Light chain of anti-TROP2 antibody
MVLQTQVFISLLLWISGAYGDIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 5 aa, synthetic construct
  REGION 1..5: note = CDRH1 of heavy chain of anti-TROP2 antibody
TAGMQ

SEQ ID NO: 4 - protein, 17 aa, synthetic construct
  REGION 1..17: note = CDRH2 of heavy chain of anti-TROP2 antibody
WINTHSGVPKYAEDFKG

SEQ ID NO: 5 - protein, 12 aa, synthetic construct
  REGION 1..12: note = CDRH3 of heavy chain of anti-TROP2 antibody
SGFGSSYWYFDV

SEQ ID NO: 6 - protein, 11 aa, synthetic construct
  REGION 1..11: note = CDRL1 of light chain of anti-TROP2 antibody
KASQDVSTAVA

SEQ ID NO: 7 - protein, 7 aa, synthetic construct
  REGION 1..7: note = CDRL2 of light chain of anti-TROP2 antibody
SASYRYT

SEQ ID NO: 8 - protein, 9 aa, synthetic construct
  REGION 1..9: note = CDRL3 of light chain of anti-TROP2 antibody
QQHYITPLT

SEQ ID NO: 9 - protein, 121 aa, synthetic construct
  REGION 1..121: note = Variable region of heavy chain of anti-TROP2 antibody
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSS

SEQ ID NO: 10 - protein, 109 aa, synthetic construct
  REGION 1..109: note = Variable region of light chain of anti-TROP2 antibody
DIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIKRT

SEQ ID NO: 11 - protein, 450 aa, synthetic construct
  REGION 1..450: note = Heavy chain of anti-TROP2 antibody
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 451 aa, synthetic construct
  REGION 1..451: note = Heavy chain of anti-TROP2 antibody
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 214 aa, synthetic construct
  REGION 1..214: note = Light chain of anti-TROP2 antibody
DIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0243773 A1
(43) Pub. Date: Aug. 20, 2026

(54) A SCORING METHOD FOR AN ANTI-TROP2 ANTIBODY-DRUG CONJUGATE THERAPY

(71) Applicants: AstraZeneca UK Limited, Cambridge (GB); DAIICHI SANKYO COMPANY, LIMITED, Tokyo (JP)

(72) Inventors: Andreas SPITZMUELLER, Bruckmuehl (DE); Guenter SCHMIDT, Munich (DE); Nicolas TRILTSCH, Munich (DE); Ansh KAPIL, II, Munich (DE)

(73) Assignees: AstraZeneca UK Limited, Cambridge (GB); DAIICHI SANKYO COMPANY, LIMITED, Tokyo (JP)

(21) Appl. No.: 18/846,571

(22) PCT Filed: Mar. 14, 2023
(86) PCT No.: PCT/IB2023/052428

§ 371 Date: Sep. 12, 2024

Related U.S. Application Data
(60) Provisional application No. 63/320,629, filed on Mar. 16, 2022.

(51) Int. Cl. G01N 33/575; A61K 47/68; C07K 16/30; G01N 1/30; G06T 7/00

(52) U.S. Cl. CPC G01N 33/5759; A61K 47/68037; A61K 47/6851; C07K 16/30; G01N 1/30; G06T 7/0012; G01N 2001/302; G01N 2333/705; G01N 2474/20; G06T 2207/10056; G06T 2207/30024

(57) ABSTRACT

A method for predicting how a cancer patient will respond to an antibody drug conjugate (ADC) therapy involves computing a predictive response score by performing statistical operations on the staining of each cancer cell in a tissue sample. The ADC includes an ADC payload and an ADC antibody that targets the trophoblast antigen 2 (TROP2) protein in the cancer cells. The tissue sample is stained using a dye linked to a diagnostic antibody that binds to the protein. Cancer cells in digital images of tissue are detected. The statistical operations are performed for each cancer cell based on the staining intensities of the dye in the membrane and/or cytoplasm of each cancer cell and/or in the membranes of neighboring cancer cells. The response of the cancer patient to the ADC therapy is predicted based on whether the results of the statistical operations exceed various clinically derived thresholds.

### CROSS-REFERENCE TO RELATED APPLICATION

This application claims priority to U.S. Provisional Application No. 63/320,629, filed on Mar. 16, 2022, which is herein incorporated by reference in its entirety.

### REFERENCE TO SEQUENCE LISTING SUBMITTED ELECTRONICALLY

The content of the electronically submitted sequence listing (Name: TROP2_402_Seqlisting.xml: Size: 19,496 bytes; and Date of Creation: Mar. 5, 2023) is herein incorporated by reference in its entirety.

### TECHNICAL FIELD

The present invention relates to a method for computing a score indicative of how a cancer patient will respond to a therapy that uses an antibody-drug conjugate having a drug conjugated to an anti-TROP2 antibody via a linker structure.

### BACKGROUND

Assessing a cancer patient's response probability to a given treatment is an essential step in determining a cancer patient's treatment regimen. Such an assessment is often based on histological analysis of tissue samples from a cancer patient and involves identifying and classifying cancers using standard grading schemes.

Immunohistochemical (IHC) staining can be used to distinguish marker-positive cells that express a particular protein from marker-negative cells that do not express the protein. IHC staining typically involves multiple dyes, which includes one or more dyes connected to protein-specific antibodies and another dye that is a counterstain. A common counterstain is hematoxylin, which labels DNA and thus stains nuclei.

A protein specific stain or biomarker can be used to identify the regions of the tissue of the cancer patient that are likely to exhibit a response to a predetermined therapy. For example, a biomarker that stains epithelial cells can help to identify the suspected tumor regions. Then other protein specific biomarkers are used to characterize the cells within the cancerous tissue. The cells stained by a specific biomarker can be identified and quantified, and subsequently a score indicating the number of positively stained cells and negatively stained cells can be visually estimated by pathologists. This score can then be compared to scores of other cancer patients that have been calculated in the same way. If the response of these other patients to a given cancer treatment is known, the pathologist can predict, based on a comparison of the score calculated for the cancer patient with the scores of the other patients, how likely the cancer patient is to respond to a given treatment. However, visual assessment by pathologists is prone to variability and subjectivity.

One promising cancer treatment involves an antibody-drug conjugate (ADC) having a drug with cytotoxicity conjugated to an antibody, whose antigen is expressed on the surface of cancer cells. The ADC binds to the antigen and undergoes cellular internalization so as to deliver the drug selectively to cancer cells and to accumulate the drug within those cancer cells and kill them. A computer-based method is sought for generating a repeatable and objective score indicating a cancer patient's response to a treatment involving a therapeutic TROP2 antibody-drug conjugate.

### SUMMARY

A method for predicting how a cancer patient will respond to a therapy involving an antibody drug conjugate (ADC) involves computing a response score based on single-cell ADC scores for each cancer cell. The ADC includes an ADC payload and an ADC antibody that targets a protein on each cancer cell. A tissue sample is immunohistochemically stained using a dye linked to a diagnostic antibody that binds to the protein on the cancer cells in the tissue sample. A digital image of the tissue sample is acquired. Image analysis is performed on the digital image to detect the cancer cells using a convolutional neural network. For each cancer cell, a single-cell ADC score is computed based on the staining intensities of the dye in the membrane and/or cytoplasm of the cancer cell and/or in the membranes and cytoplasms of other cancer cells that are closer than a predefined distance to the cancer cell. A response score is generated that predicts the response of the cancer patient to the ADC therapy by aggregating all single-cell ADC scores of the tissue sample using a statistical operation. Patients having a response score higher than a predetermined threshold are recommended for a therapy involving the ADC.

In one embodiment, a method of predicting a response of a cancer patient to an ADC involves computing single-cell ADC scores. A tissue sample of the cancer patient is immunohistochemically stained using a dye linked to a diagnostic antibody. The ADC includes an ADC payload and an ADC antibody that targets a trophoblast cell-surface antigen 2 (TROP2) protein on cancer cells. The diagnostic antibody binds to the TROP2 protein on cancer cells in the tissue sample. A digital image of the tissue sample is acquired, and cancer cells in the digital image are detected using image analysis. For each cancer cell, a single-cell ADC score is computed based on the staining intensity of the dye in the membrane. The single-cell ADC score may also optionally be based on the staining intensity of the dye in the cytoplasm of the cancer cell, as well as on the staining intensities of the dye in the membranes and the cytoplasms of other cancer cells that are closer than a predefined distance to the cancer cell. The staining intensity of each membrane is computed based on the average optical density of a brown diaminobenzidine (DAB) signal in pixels of the membrane, and the staining intensity of each cytoplasm is computed based on the average optical density of the brown DAB signal in pixels of the cytoplasm. The resulting Quantitative Continuous Score (QCS), which is indicative of the response of the cancer patient to the ADC, is generated by aggregating all single-cell ADC scores of the tissue sample using a statistical operation. The aggregating of all single-cell ADC scores is performed by determining the mean, determining the median, or determining a quantile with a predefined percentage. In another aspect, the aggregation of all single-cell ADC scores involves a threshold operation using a predefined threshold. All cells having a single-cell ADC score larger than the predefined threshold are labeled single-cell ADC positive. The aggregation is performed by determining the number of single-cell ADC positive cells divided by the number of all cancer cells. In one example, the QCS score is a continuous spatial proximity score.

In another embodiment, a method of identifying a cancer patient for treatment with an ADC involves determining a binary spatial proximity score for a tissue sample of the cancer patient. The ADC includes an ADC payload and an ADC antibody that targets the TROP2 protein on cancer cells. A tissue sample from the cancer patient is immunohistochemically stained using a dye linked to a diagnostic antibody that binds to the TROP2 protein on the cancer cells in the tissue sample. A digital image of the tissue sample is acquired, and cancer cells in the digital image are detected using a convolutional neural network. Cancer cells are detected in the digital image. For each cancer cell, the mean optical density of staining by the dye in the cell membrane is determined. Each cancer cell is identified as being either optical-density positive if the mean optical density of the cancer cell is equal to or greater than an optical density threshold or optical-density negative if the mean optical density of the cancer cell is less than the optical density threshold. The tissue sample is identified as being proximity positive if the total percentage of cancer cells that are either optical density positive or optical density negative but within a predefined distance of an optical density positive cancer cell exceeds a proximity percentage threshold. The cancer patient is identified as one who will likely benefit from administration of the ADC if the tissue sample is proximity positive. A therapy involving the ADC is recommended to the cancer patient when the tissue sample is proximity positive. In another aspect, a method of treating cancer involves administering the ADC to the cancer patient if the tissue sample is proximity positive.

In another embodiment, a method of identifying a cancer patient for treatment with an ADC involves determining the normalized staining of the cell membrane of stained cancer cells. The ADC includes an ADC payload and an ADC antibody. A tissue sample of the cancer patient is immunohistochemically stained using a dye linked to a diagnostic antibody. Both the diagnostic antibody and the ADC antibody target the TROP2 protein on cancer cells. A digital image of the tissue sample is acquired. Image analysis is performed on the digital image to detect the cancer cells using a convolutional neural network. For each cancer cell, the mean optical density of staining by the dye in the membrane of the cancer cell is determined, and the mean optical density of staining by the dye in the cytoplasm of the cancer cell is determined. The normalized membrane optical density (nmOD) for each cancer cell in the digital image is computed, which equals the mean optical density of staining of the membrane divided by the sum of the mean optical density of staining of the membrane plus the mean optical density of staining of the cytoplasm. Each cancer cell is identified as being nmOD positive if the nmOD of the cancer cell is equal to or less than a nmOD threshold. The tissue sample is identified as being nmOD positive if the percentage of cancer cells that are nmOD positive is equal to or greater than a nmOD percentage threshold. The cancer patient is identified as one who will likely benefit from administration of the ADC if the tissue sample is nmOD positive. A therapy involving the ADC is recommended to the cancer patient when the tissue sample is nmOD positive. In another aspect, a method of treating cancer involves administering the ADC to the cancer patient if the tissue sample is nmOD positive.

In another embodiment, a method of identifying a cancer patient for treatment with an ADC is based on both the spatial proximity of variously stained cancer cells and on the normalized staining of the cell membrane of stained cancer cells. The ADC includes an ADC payload and an ADC antibody. A tissue sample of the cancer patient is immunohistochemically stained using a dye linked to a diagnostic antibody. Both the diagnostic antibody and the ADC antibody target the TROP2 protein on cancer cells. A digital image of the tissue sample is acquired. Image analysis is performed on the digital image to detect the cancer cells using a convolutional neural network. For each cancer cell, the mean optical density of membrane staining and the mean optical density of cytoplasm staining are determined.

A normalized membrane optical density (nmOD) is computed for each cancer cell in the digital image, which equals the mean optical density of membrane staining divided by the sum of the mean optical density of membrane staining plus the mean optical density of cytoplasm staining. Each cancer cell is identified as being nmOD positive if the nmOD of the cancer cell is equal to or less than a nmOD threshold. The tissue sample is identified as being nmOD positive if the percentage of cancer cells that are nmOD positive is equal to or greater than a nmOD percentage threshold. Each cancer cell is identified as being either optical-density positive if the mean optical density of membrane staining is equal to or greater than an optical density threshold or optical-density negative if the mean optical density of membrane staining is less than the optical density threshold. The tissue sample is identified as being proximity positive if the total percentage of cancer cells that are either optical-density positive or optical-density negative but within a predefined distance of an optical-density positive cancer cell exceeds a proximity percentage threshold. The cancer patient is identified as one who will likely benefit from administration of the ADC if the tissue sample is both nmOD positive and proximity positive. A therapy involving the ADC is recommended to the cancer patient when the tissue sample is both nmOD positive and proximity positive. In another aspect, a method of treating cancer involves administering the ADC to the cancer patient if the tissue sample is both nmOD positive and proximity positive.

Other embodiments and advantages are described in the detailed description below. This summary does not purport to define the invention. The invention is defined by the claims.

### BRIEF DESCRIPTION OF THE DRAWINGS

The accompanying drawings, where like numerals indicate like components, illustrate embodiments of the invention.

FIG. 1 shows the amino acid sequence (SEQ ID No: 1) of a heavy chain of the anti-TROP2 antibody.

FIG. 2 shows the amino acid sequence (SEQ ID No: 2) of a light chain of the anti-TROP2 antibody.

FIG. 3 shows the amino acid sequence (SEQ ID No: 3) of CDRH1 of the anti-TROP2 antibody.

FIG. 4 shows the amino acid sequence (SEQ ID No: 4) of CDRH2 of the anti-TROP2 antibody.

FIG. 5 shows the amino acid sequence (SEQ ID No: 5) of CDRH3 of the anti-TROP2 antibody.

FIG. 6 shows the amino acid sequence (SEQ ID No: 6) of CDRL1 of the anti-TROP2 antibody.

FIG. 7 shows the amino acid sequence (SEQ ID No: 7) of CDRL2 of the anti-TROP2 antibody.

FIG. 8 shows the amino acid sequence (SEQ ID No: 8) of CDRL3 of the anti-TROP2 antibody.

FIG. 9 shows the amino acid sequence (SEQ ID No: 9) of a heavy chain variable region of the anti-TROP2 antibody.

FIG. 10 shows the amino acid sequence (SEQ ID No: 10) of a light chain variable region of the anti-TROP2 antibody.

FIG. 11 shows the amino acid sequence (SEQ ID No: 11) of a heavy chain of the anti-TROP2 antibody.

FIG. 12 illustrates the anti-TROP2 antibody-drug conjugate datopotamab deruxtecan with four drug-linker units.

FIG. 13 is a flowchart of steps by which an analysis system analyzes digital images of tissue from a cancer patient and predicts how the cancer patient will likely respond to a therapy involving an anti-TROP2 antibody-drug conjugate.

FIG. 14 shows digital images illustrating the image analysis process of step 12 of FIG. 13.

FIG. 15 illustrates image analysis steps in which nucleus objects of cancer cells are detected.

FIG. 16 illustrates image analysis steps in which nucleus objects are used to detect membranes.

FIG. 17 is a screenshot of the results of the image analysis steps in an image analysis software environment.

FIG. 18 shows a sample calculation of a binary spatial proximity score for ten exemplary cells based on cell separation to reflect the uptake of the ADC payload into neighboring cells.

FIG. 19 illustrates the mechanism by which an anti-TROP2 ADC therapy kills cancer cells.

FIG. 20 shows sample quantitative results of staining intensities from image analysis using gray values of membrane and cytoplasm pixels.

FIG. 21 lists the exemplary quantitative amounts of staining on the membranes and in the cytoplasms of the image of FIG. 20.

FIG. 22 illustrates the calculation of a continuous spatial proximity score for each of the three cells shown in FIG. 21 based on cell separation to reflect the uptake of the ADC payload into neighboring cells.

FIG. 23 shows a formula by which a continuous spatial proximity score is calculated for each cancer cell.

FIG. 24 is a plot showing the response to the ADC administered to 115 patients of a clinical trial in terms of tumor growth/shrinkage compared to the response score of the method of FIG. 13 (bystander_memb(meanOD)_binary_r50_cut25). In the plot, patients are denoted as having progressive disease (PD), stable disease (SD), partial response (PR), or being non-evaluable (NE).

FIG. 25 is a graph of Kaplan-Meier curves of progression-free survival for two groups of the 115 patients that were stratified into QCS Positive and QCS Negative patients using the binary spatial proximity score of the method of FIG. 13. The graph shows the objective response rate (ORR) of the QCS Positive and QCS Negative patients.

FIG. 26 shows spider plots of tumor shrinkage over time for 88 bSPS positive patients and the 27 bSPS negative patients of the Kaplan-Meier curves of FIG. 25.

FIG. 27 is a waterfall bar graph showing the clinical response of 105 of the 115 patients of FIG. 25 ordered from greatest tumor growth to greatest tumor shrinkage.

FIG. 28 is a flowchart of steps of a novel method for predicting the response of a cancer patient to an ADC based on the normalized membrane optical density of each cancer cell.

FIG. 29 is a plot showing the response to the ADC administered to the 115 patients in terms of tumor growth/shrinkage compared to the response score of the method of FIG. 28 (memb(meanOD)/(memb(meanOD)+cyto(meanOD))_q5). The plot identifies patients as having progressive disease (PD), stable disease (SD), partial response (PR) or as being non-evaluable (NE).

FIG. 30 is a graph of Kaplan-Meier curves of progression-free survival for two groups of the 115 patients that were stratified into QCS Positive and QCS Negative patients using the response score of the method of FIG. 28. The graph shows the objective response rate (ORR) of the QCS Positive and QCS Negative patients.

FIG. 31 shows spider plots of tumor shrinkage over time for 84 QCS Positive patients and the 31 QCS Negative patients of the Kaplan-Meier curves of FIG. 30.

FIG. 32 is a waterfall bar graph showing the clinical response of 105 of the 115 patients of FIG. 30 ordered from greatest tumor growth to greatest tumor shrinkage.

FIG. 33 is a flowchart of steps of a novel method for predicting the response of a cancer patient to an ADC based on a combination of the binary spatial proximity score and the normalized membrane optical density score.

FIG. 34 is a graph of Kaplan-Meier curves of progression-free survival for two groups of the 115 patients that were stratified into QCS Positive and QCS Negative patients using the response score of the method of FIG. 33. The graph shows the objective response rate (ORR) of the QCS Positive and QCS Negative patients.

FIG. 35 shows spider plots of tumor shrinkage over time for 80 QCS Positive patients and the 35 QCS Negative patients of the Kaplan-Meier curves of FIG. 34.

FIG. 36 is a waterfall bar graph showing the clinical response of 105 of the 115 patients of FIG. 34 ordered from greatest tumor growth to greatest tumor shrinkage.

### DETAILED DESCRIPTION

The present invention relates to novel methods for predicting a response of a cancer patient to an antibody drug conjugate (ADC) including an ADC antibody that targets the calcium signal transducer trophoblast antigen 2 (TROP2) protein on cancer cells, wherein the response is predicted based on statistical operations using the measured optical density of staining by a dye linked to a diagnostic antibody that also targets the TROP2 protein. Another aspect of the invention relates to a method for identifying cancer patients for treatment with the ADC based on a QCS score. Another aspect of the invention relates to identifying a cancer patient who will exhibit a predetermined response to the ADC. Yet another aspect of the invention relates to a method of treating a cancer patient by administering a therapy involving the ADC based on a quantitative continuous score (QCS) computed using one of the statistical operations.

A first embodiment relates to identifying a cancer patient who will likely benefit from administration of the ADC based on a binary spatial proximity score equaling a percentage of cancer cells in the patient's tissue sample that either have a mean optical density of staining above an optical density threshold or have a mean optical density of staining below the optical density threshold but are disposed within a predefined distance of a cell whose mean optical density of staining lies above the optical density threshold.

A second embodiment relates to identifying a cancer patient who will likely benefit from administration of the ADC based on a normalized membrane optical density for each cancer cell that equals the mean optical density of membrane staining divided by the sum of the mean optical density of membrane staining plus the mean optical density of cytoplasm staining.

A third embodiment relates to identifying a cancer patient who will likely benefit from administration of the ADC based on both the binary spatial proximity score an on the normalized membrane optical density. The cancer patient is identified as likely benefiting from the ADC if both the binary spatial proximity score exceeds a proximity percentage threshold, and the percentage of cancer cells that are normalized membrane positive is equal to or greater than a normalized percentage threshold.

### I. Definitions

To facilitate an understanding of the present invention, a number of terms and phrases are defined below. The term “cancer” is used to have the same meaning as that of the term “tumor”. In the present disclosure, “TROP2” is synonymous with the calcium signal transducer trophoblast antigen 2 transmembrane protein. In the present disclosure, the term “TROP2 protein” is used in the same meaning as TROP2. The expression of the TROP2 protein can be detected using a method well known to those skilled in the art, such as immunohistochemistry (IHC) or immunofluorescence (IF).

In the present invention, “anti-TROP2 antibody” means an antibody that specifically binds to TROP2. The anti-TROP2 antibody has an activity of binding to TROP2 and is thereby internalized into TROP2-expressing cells, such that after exhibiting the activity of binding to TROP2, the antibody moves into the TROP2 expressing cells. The anti-TROP2 antibody targets tumor cells, binds to the tumor cells, internalizes into the tumor cells, exhibits cytocidal activity against the tumor cells, and can be conjugated with a drug having antitumor activity via a linker to form an antibody-drug conjugate.

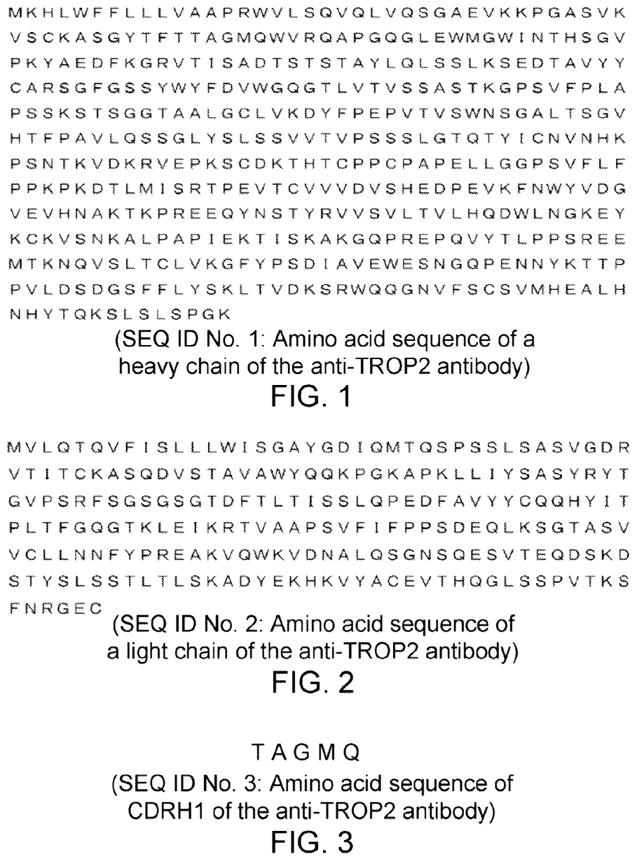

FIG. 1 shows the amino acid sequence (SEQ ID No. 1) of a heavy chain of an exemplary anti-TROP2 antibody. The heavy chain has the signal sequence (1-19), the variable region (20-140), and the constant region (141-470).

FIG. 2 shows the amino acid sequence (SEQ ID No. 2) of a light chain of an exemplary anti-TROP2 antibody. The light chain has the signal sequence (1-20), the variable region (21-129), and the constant region (130-234).

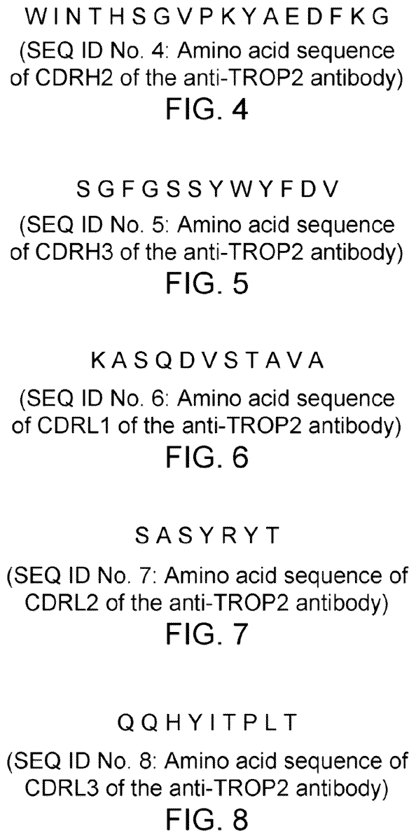

FIG. 3 shows the amino acid sequence (SEQ ID No: 3) of CDRH1 of an exemplary anti-TROP2 antibody, FIG. 4 shows the amino acid sequence (SEQ ID No: 4) of CDRH2 of an exemplary anti-TROP2 antibody, and FIG. 5 shows the amino acid sequence (SEQ ID No: 5) of CDRH3 of an exemplary anti-TROP2 antibody.

FIG. 6 shows the amino acid sequence (SEQ ID No: 6) of CDRL1 of an exemplary anti-TROP2 antibody, FIG. 7 shows the amino acid sequence (SEQ ID No: 7) of CDRL2 (SAS) of an exemplary anti-TROP2 antibody, and FIG. 8 shows the amino acid sequence (SEQ ID No: 8) of CDRL3 of an exemplary anti-TROP2 antibody.

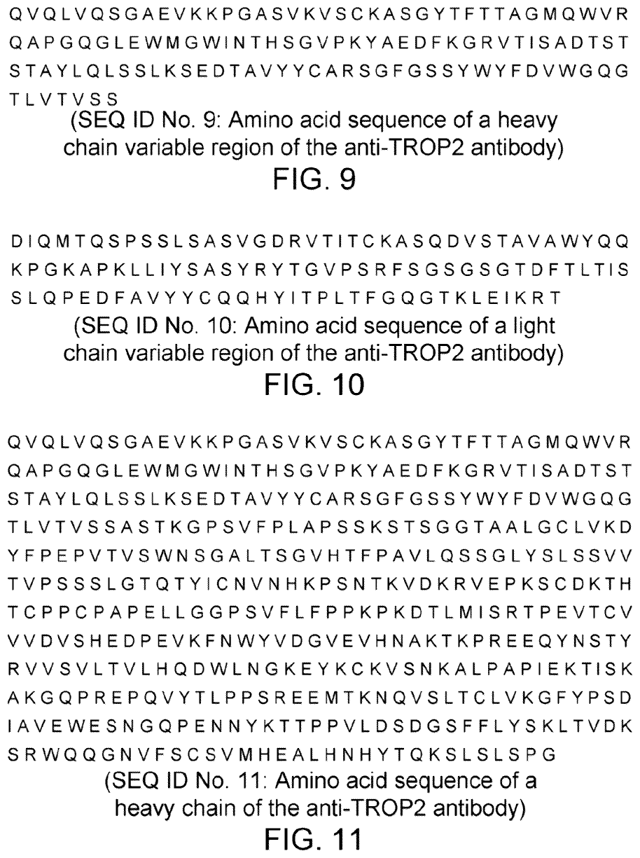

FIG. 9 shows the amino acid sequence (SEQ ID No: 9) of a heavy chain variable region of an exemplary anti-TROP2 antibody, and FIG. 10 shows the amino acid sequence (SEQ ID No: 10) of a light chain variable region of an exemplary anti-TROP2 antibody. FIG. 11 shows the amino acid sequence (SEQ ID No: 11) of another heavy chain of an exemplary anti-TROP2 antibody.

An anti-TROP2 antibody of the anti-TROP2 antibody-drug conjugate used in the present invention is preferably an antibody comprising a heavy chain comprising CDRH1 consisting of an amino acid sequence represented by SEQ ID No. 3 (an amino acid sequence consisting of amino acid residues 50 through 54 of SEQ ID No. 1), CDRH2 consisting of an amino acid sequence represented by SEQ ID No. 4 (an amino acid sequence consisting of amino acid residues 69 through 85 of SEQ ID No. 1) and CDRH3 consisting of an amino acid sequence represented by SEQ ID No. 5 (an amino acid sequence consisting of amino acid residues 118 through 129 of SEQ ID No. 1), and a light chain comprising CDRL1 consisting of an amino acid sequence represented by SEQ ID No. 6 (an amino acid sequence consisting of amino acid residues 44 through 54 of SEQ ID No. 2), CDRL2 consisting of an amino acid sequence represented by SEQ ID No. 7 (an amino acid sequence consisting of amino acid residues 70 to 76 of SEQ ID No. 2) and CDRL3 consisting of an amino acid sequence represented by SEQ ID No. 8 (an amino acid sequence consisting of amino acid residues 109 through 117 of SEQ ID No. 2), and more preferably an antibody comprising a heavy chain variable region consisting of an amino acid sequence represented by SEQ ID NO: 9 (an amino acid sequence consisting of amino acid residues 20 through 140 of SEQ ID No. 1) and a light chain variable region consisting of an amino acid sequence represented by SEQ ID No. 10 (an amino acid sequence consisting of amino acid residues 21 through 129 of SEQ ID No. 2). In other preferred embodiments, the antibody comprises a heavy chain consisting of an amino acid sequence represented by SEQ ID No. 11 (an amino acid sequence consisting of amino acid residues 20 through 469 of SEQ ID No. 1) and a light chain consisting of an amino acid sequence represented by amino acid residues 21-234 of SEQ ID No. 2. In other embodiments, the antibody comprises a heavy chain consisting of an amino acid sequence represented by amino acid residues 20-470 of SEQ ID No. 1 and a light chain consisting of an amino acid sequence represented by amino acid residues 21-234 of SEQ ID No. 2.

In the present invention, the term “QCS Positive” (QCS+) refers to cancer that is likely to show a response to an anti-TROP2 ADC therapy. The term “QCS Negative” (QCS−) refers to cancer that is unlikely to show a response to an anti-TROP2 ADC therapy. The acronym QCS stands for Quantitative Continuous Score. The result of the novel predictive method of the present invention is generally referred to as a Quantitative Continuous Score and may be a response score, a treatment score or an indication of predicted survival time. The QCS score is obtained by performing statistical operations on all of the single-cell ADC scores obtained for a patient. Applying a predetermined threshold to the QCS scores discriminates between “QCS Positive” and “QCS Negative” patients. Stratifying the cancer patients into the QCS Positive and QCS Negative groups enables the identification of those QCS+ patients who likely benefit from the therapy which involves the ADC.

### II. The Anti-TROP2 Antibody-Drug Conjugate

An exemplary antibody-drug conjugate used in the present disclosure is an antibody-drug conjugate in which a drug-linker represented by the following formula:

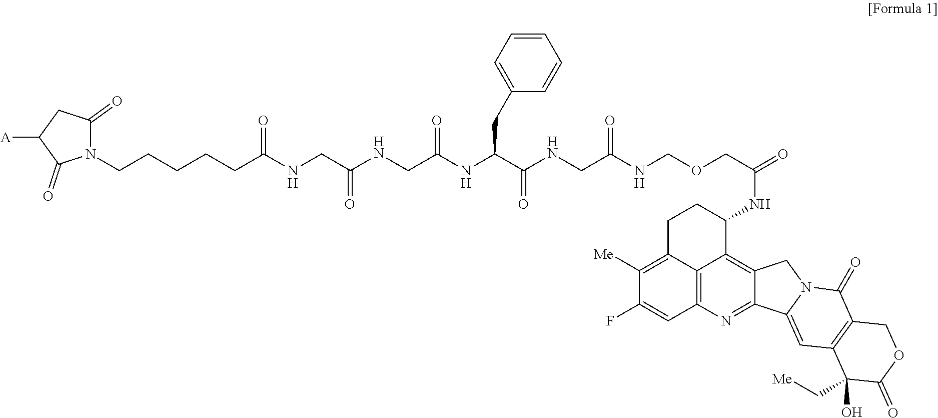

wherein A represents the connecting position to an antibody and is conjugated to an anti-TROP2 antibody via a thioether bond.

In the present disclosure, the partial structure consisting of a linker and a drug in the antibody-drug conjugate is referred to as a “drug-linker”. The drug-linker is connected to a thiol group (in other words, the sulfur atom of a cysteine residue) formed at an interchain disulfide bond site (two sites between heavy chains, and two sites between a heavy chain and a light chain) in the antibody.

The drug-linker of the present disclosure may include exatecan (IUPAC name: (1S,9S)-1-amino-9-ethyl-5-fluoro-1,2,3,9,12,15-hexahydro-9-hydroxy-4-methyl-10H, 13H-benzo[de]pyrano[3′,4′: 6,7]indolizino[1,2-b]quinolin-10,13-dione, (also expressed as chemical name: (1S,9S)-1-amino-9-ethyl-5-fluoro-2,3-dihydro-9-hydroxy-4-methyl-1H,12H-benzo[de]pyrano[3′,4′: 6,7]indolizino[1,2-b]quinolin-10,13 (9H,15H)-dione)), which is a topoisomerase I inhibitor, as a component. Exatecan is a camptothecin derivative having an antitumor effect, represented by the following formula:

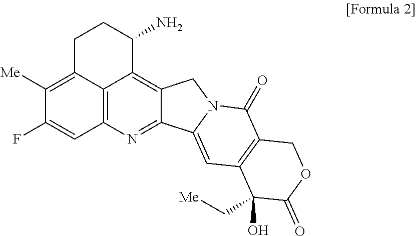 [SMILES: CC[C@@]1(O)C(=O)OCc2c1cc1n(c2=O)Cc2c-1nc1cc(F)c(C)c3c1c2[C@@H](N)CC3]

An exemplary anti-TROP2 antibody-drug conjugate used in the present disclosure can be also represented by the following formula:

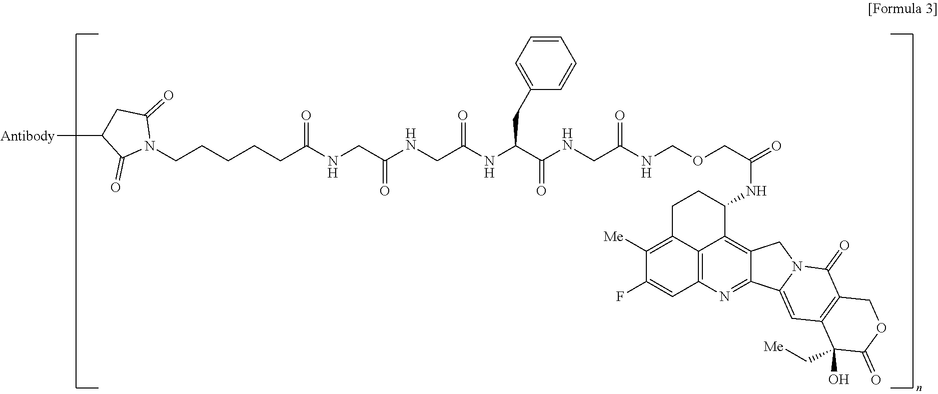

Here, the drug-linker is conjugated to an anti-TROP2 antibody (‘Antibody-’) via a thioether bond. The meaning of n is the same as that of what is called the average number of conjugated drug molecules (DAR; Drug-to-Antibody Ratio), and indicates the average number of units of the drug-linker conjugated per antibody molecule.

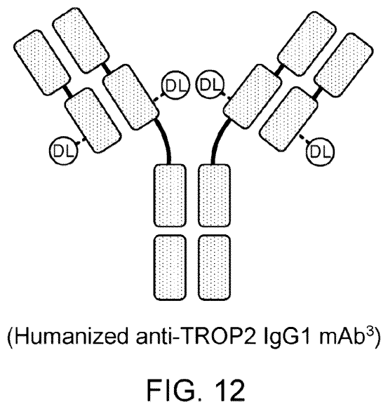

FIG. 12 illustrates a preferred anti-TROP2 antibody-drug conjugate datopotamab deruxtecan (DS-1062), with four drug-linker units designated as “DL”. The datopotamab portion of datopotamab deruxtecan shown in FIG. 12 is humanized anti-TROP2 IgG1 mAb, wherein IgG1 indicates the isotype of the anti-TROP2 antibody.

After migrating into cancer cells, the preferred anti-TROP2 antibody-drug conjugate used in the present disclosure is cleaved at the linker portion to release a compound represented by the following formula:

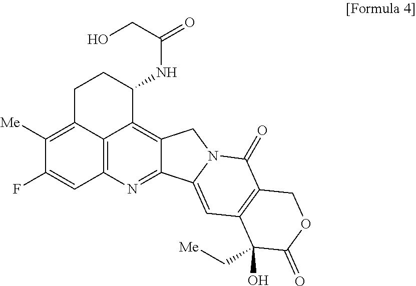 [SMILES: CC[C@@]1(O)C(=O)OCc2c1cc1n(c2=O)Cc2c-1nc1cc(F)c(C)c3c1c2[C@@H](NC(=O)CO)CC3]

The compound shown above is the primary source of antitumor activity of the preferred anti-TROP2 antibody-drug conjugate used in the present invention, and has a topoisomerase I inhibitory effect.

The preferred anti-TROP2 antibody-drug conjugate used in the present invention also has a bystander effect in which the anti-TROP2 antibody-drug conjugate is internalized into cancer cells that express the target protein TROP2, and the compound shown above then also exerts an antitumor effect on neighboring cancer cells that do not express the target protein TROP2.

### III. The Anti-TROP2 Antibody in the Antibody-Drug Conjugate

The anti-TROP2 antibody in the antibody-drug conjugate used in the present invention may be derived from any species and is preferably an antibody derived from a human, a rat, a mouse, or a rabbit. In cases when the antibody is derived from species other than human species, it is preferably chimerized or humanized using a well known technique. The antibody of the present invention may be a polyclonal antibody or a monoclonal antibody and is preferably a monoclonal antibody.

The antibody in the antibody-drug conjugate used in the present invention is an antibody preferably having the characteristic of being able to target cancer cells, and is preferably an antibody possessing, for example, the property of being able to recognize a cancer cell, the property of being able to bind to a cancer cell, the property of being internalized in a cancer cell, and/or cytocidal activity against cancer cells.

The binding activity of the antibody against cancer cells can be confirmed using flow cytometry. The internalization of the antibody into tumor cells can be confirmed using (1) an assay of visualizing an antibody incorporated in cells under a fluorescence microscope using a secondary antibody (fluorescently labeled) binding to the therapeutic antibody (Cell Death and Differentiation (2008) 15, 751-761), (2) an assay of measuring a fluorescence intensity incorporated in cells using a secondary antibody (fluorescently labeled) binding to the therapeutic antibody (Molecular Biology of the Cell, Vol. 15, 5268-5282 December 2004), or (3) a Mab-ZAP assay using an immunotoxin binding to the therapeutic antibody wherein the toxin is released upon incorporation into cells to inhibit cell growth (Bio Techniques 28:162-165, January 2000). As the immunotoxin, a recombinant complex protein of a diphtheria toxin catalytic domain and protein G may be used.

The antitumor activity of the antibody can be confirmed in vitro by determining inhibitory activity against cell growth. For example, a cancer cell line overexpressing a target protein for the antibody is cultured, and the antibody is added at varying concentrations into the culture system to determine inhibitory activity against focus formation, colony formation, and spheroid growth. The antitumor activity can be confirmed in vivo, for example, by administering the antibody to a nude mouse with a transplanted cancer cell line highly expressing the target protein, and determining changes in the cancer cells.

Since the compound conjugated in the antibody-drug conjugate exerts an antitumor effect, it is preferred but not essential that the antibody itself should have an antitumor effect. For the purpose of specifically and selectively exerting the cytotoxic activity of the antitumor compound against cancer cells, it is important and also preferred that the antibody should have the property of being internalized to migrate into cancer cells.

The antibody in the antibody-drug conjugate used in the present invention can be obtained by a procedure known in the art. For example, the antibody of the present invention can be obtained using a method usually carried out in the art, which involves immunizing animals with an antigenic polypeptide and collecting and purifying antibodies produced in vivo. The origin of the antigen is not limited to humans, and the animals may be immunized with an antigen derived from a non-human animal such as a mouse, a rat and the like. In this case, the cross-reactivity of antibodies binding to the obtained heterologous antigen with human antigens can be tested to screen for an antibody applicable to a human disease.

Alternatively, antibody-producing cells which produce antibodies against the antigen can be fused with myeloma cells according to a method known in the art (for example, Kohler and Milstein, Nature (1975) 256, p. 495-497; Kennet, R. ed., Monoclonal Antibodies, p. 365-367, Plenum Press, N.Y. (1980)), to establish hybridomas, from which monoclonal antibodies can in turn be obtained.

The antigen can be obtained by genetically engineering host cells to produce a gene encoding the antigenic protein. Specifically, vectors that permit expression of the antigen gene are prepared and transferred to host cells so that the gene is expressed. The antigen thus expressed can be purified. The antibody can also be obtained by a method of immunizing animals with the above-described genetically engineered antigen-expressing cells or a cell line expressing the antigen.

The antibody in the antibody-drug conjugate used in the present invention is preferably a recombinant antibody obtained by artificial modification for the purpose of decreasing heterologous antigenicity to humans such as a chimeric antibody or a humanized antibody, or is preferably an antibody having only the gene sequence of an antibody derived from a human, that is, a human antibody. These antibodies can be produced using a known method.

As the chimeric antibody, an antibody in which antibody variable and constant regions are derived from different species, for example, a chimeric antibody in which a mouse- or rat-derived antibody variable region is connected to a human-derived antibody constant region can be exemplified (Proc. Natl. Acad. Sci. USA, 81, 6851-6855, (1984)).

As the humanized antibody, an antibody obtained by integrating only the complementarity determining region (CDR) of a heterologous antibody into a human-derived antibody (Nature (1986) 321, pp. 522-525), an antibody obtained by grafting a part of the amino acid residues of the framework of a heterologous antibody as well as the CDR sequence of the heterologous antibody to a human antibody by a CDR-grafting method (WO 90/07861), and an antibody humanized using a gene conversion mutagenesis strategy (U.S. Pat. No. 5,821,337) can be exemplified.

As the human antibody, an antibody generated by using a human antibody-producing mouse having a human chromosome fragment including genes of a heavy chain and light chain of a human antibody (see Tomizuka, K. et al., Nature Genetics (1997) 16, p. 133-143; Kuroiwa, Y. et. al., Nucl. Acids Res. (1998) 26, p. 3447-3448; Yoshida, H. et. al., Animal Cell Technology: Basic and Applied Aspects vol. 10, p. 69-73 (Kitagawa, Y., Matsuda, T. and Iijima, S. eds.), Kluwer Academic Publishers, 1999; Tomizuka, K. et. al., Proc. Natl. Acad. Sci. USA (2000) 97, p. 722-727, etc.) can be exemplified. As an alternative, an antibody obtained by phage display, the antibody being selected from a human antibody library (see Wormstone, I. M. et. al, Investigative Ophthalmology & Visual Science. (2002) 43 (7), p. 2301-2308; Carmen, S. et. al., Briefings in Functional Genomics and Proteomics (2002), 1 (2), p. 189-203; Siriwardena, D. et. al., Ophthalmology (2002) 109 (3), p. 427-431, etc.) can be exemplified.

In the antibody in the antibody-drug conjugate used in present invention, modified variants of the antibody are also included. The modified variant refers to a variant obtained by subjecting the antibody according to the present invention to chemical or biological modification. Examples of the chemically modified variant include variants including a linkage of a chemical moiety to an amino acid skeleton, variants including a linkage of a chemical moiety to an N-linked or O-linked carbohydrate chain, etc. Examples of the biologically modified variant include variants obtained by post-translational modification (such as N-linked or O-linked glycosylation, N- or C-terminal processing, deamidation, isomerization of aspartic acid, or oxidation of methionine), and variants in which a methionine residue has been added to the N terminus by being expressed in a prokaryotic host cell. Further, an antibody labeled so as to enable the detection or isolation of the antibody or an antigen according to the present invention, for example, an enzyme-labeled antibody, a fluorescence-labeled antibody, and an affinity-labeled antibody are also included in the meaning of the modified variant. Such a modified variant of the antibody according to the present invention is useful for improving the stability and blood retention of the antibody, reducing the antigenicity thereof, detecting or isolating an antibody or an antigen, and so on.

Further, by regulating the modification of a glycan which is linked to the antibody according to the present invention (glycosylation, defucosylation, etc.), it is possible to enhance antibody-dependent cellular cytotoxic activity. As the technique for regulating the modification of a glycan of antibodies, International Publication No. WO 99/54342, International Publication No. WO 00/61739, International Publication No. WO 02/31140, International Publication No. WO 2007/133855, International Publication No. WO 2013/120066, etc. are known. However, the technique is not limited thereto. In the antibody according to the present invention, antibodies in which the modification of a glycan is regulated are also included.

It is known that a lysine residue at the carboxyl terminus of the heavy chain of an antibody produced in a cultured mammalian cell is deleted (Journal of Chromatography A, 705:129-134 (1995)), and it is also known that two amino acid residues (glycine and lysine) at the carboxyl terminus of the heavy chain of an antibody produced in a cultured mammalian cell are deleted and a proline residue newly located at the carboxyl terminus is amidated (Analytical Biochemistry, 360:75-83 (2007)). However, such deletion and modification of the heavy chain sequence do not affect the antigen-binding affinity and the effector function (complement activation, antibody-dependent cellular cytotoxicity, etc.) of the antibody. Therefore, in the antibody according to the present invention, antibodies subjected to such modification and functional fragments of the antibody are also included, and deletion variants in which one or two amino acids have been deleted at the carboxyl terminus of the heavy chain, variants obtained by amidation of the deletion variants (for example, a heavy chain in which the carboxyl terminal proline residue has been amidated) and the like are also included. The type of deletion variant having a deletion at the carboxyl terminus of the heavy chain of the antibody according to the present invention is not limited to the above variants as long as the antigen-binding affinity and the effector function are conserved. The two heavy chains constituting the antibody according to the present invention may be of one type selected from the group consisting of a full-length heavy chain and the above-described deletion variant, or may be of two types in combination selected therefrom. The ratio of the amount of each deletion variant can be affected by the type of cultured mammalian cells that produce the antibody according to the present invention and the culture conditions; however, an antibody in which one amino acid residue at the carboxyl terminus has been deleted in both of the two heavy chains in the antibody according to the present invention can be preferably exemplified.

As isotypes of the antibody according to the present invention, for example, IgG (IgG1, IgG2, IgG3, IgG4) can be exemplified. Preferably, IgG1 or IgG2 can be exemplified.

In the present invention, the term “anti-TROP2 antibody” refers to an antibody which binds specifically to TROP2 (TACSTD2: Tumor-associated calcium signal transducer 2; EGP-1), and preferably has an activity of internalization in TROP2-expressing cells by binding to TROP2.

Examples of the anti-TROP2 antibody include hTINA1-H1L1 (WO 2015/098099).

### IV. Producing the Anti-TROP2 Antibody-Drug Conjugate

A drug-linker intermediate for use in the production of the antibody-drug conjugate according to the present invention is represented by the following formula.

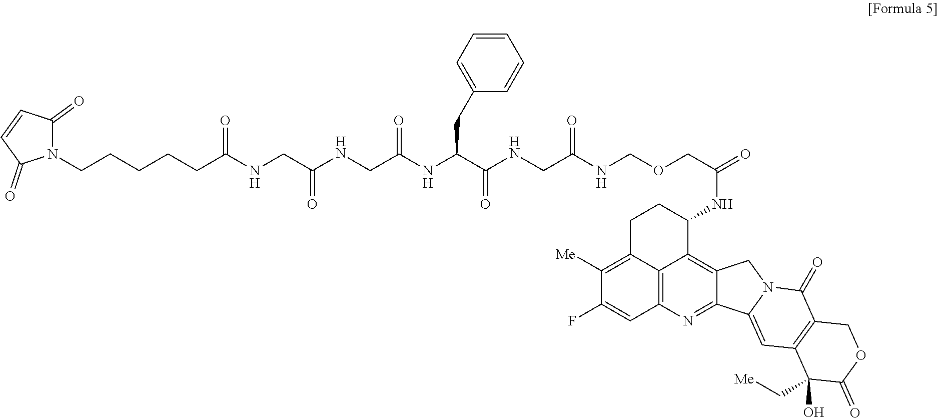 [SMILES: CC[C@@]1(O)C(=O)OCc2c1cc1n(c2=O)Cc2c-1nc1cc(F)c(C)c3c1c2[C@@H](NC(=O)COCNC(=O)CNC(=O)[C@H](Cc1ccccc1)NC(=O)CNC(=O)CNC(=O)CCCCCN1C(=O)C=CC1=O)CC3]

The drug-linker intermediate can be expressed as the chemical name N-[6-(2,5-dioxo-2,5-dihydro-1H-pyrrol-1-yl) hexanoyl]glycylglycyl-L-phenylalanyl-N-[(2-{[(1S,9S)-9-ethyl-5-fluoro-9-hydroxy-4-methyl-10,13-dioxo-2,3,9,10,13,15-hexahydro-1H,12H-benzo[de]pyrano[3′,4′: 6,7]indolizino[1,2-b]quinolin-1-yl]amino}-2-oxoethoxy)methyl]glycinamide, and can be produced with reference to descriptions in WO 2014/057687, WO 2015/098099, WO 2015/115091, WO 2015/155998, WO 2019/044947, and so on.

The antibody-drug conjugate used in the present invention can be produced by reacting the above-described drug-linker intermediate and an anti-TROP2 antibody having a thiol group (alternatively referred to as a sulfhydryl group).

The anti-TROP2 antibody having a sulfhydryl group can be obtained by a method well known in the art (Hermanson, G. T, Bioconjugate Techniques, pp. 56-136, pp. 456-493, Academic Press (1996)). For example, by using 0.3 to 3 molar equivalents of a reducing agent such as tris(2-carboxyethyl) phosphine hydrochloride (TCEP) per interchain disulfide within the antibody and reacting with the antibody in a buffer solution containing a chelating agent such as ethylenediamine tetraacetic acid (EDTA), an antibody having a sulfhydryl group with partially or completely reduced interchain disulfides within the antibody can be obtained.

Further, by using 2 to 20 molar equivalents of the drug-linker intermediate per the antibody having a sulfhydryl group, an antibody-drug conjugate in which 2 to 8 drug molecules are conjugated per antibody molecule can be produced.

The average number of conjugated drug molecules per antibody molecule of the antibody-drug conjugate produced can be determined, for example, by a method of calculation based on measurement of UV absorbance for the antibody-drug conjugate and the conjugation precursor thereof at two wavelengths of 280 nm and 370 nm (UV method), or a method of calculation based on quantification through HPLC measurement for fragments obtained by treating the antibody-drug conjugate with a reducing agent (HPLC method).

Conjugation between the antibody and the drug-linker intermediate and calculation of the average number of conjugated drug molecules per antibody molecule of the antibody-drug conjugate can be performed with reference to descriptions in WO 2015/098099 and WO 2017/002776, for example.

In the present invention, the term “anti-TROP2 antibody-drug conjugate” refers to an antibody-drug conjugate such that the antibody in the antibody-drug conjugate according to the invention is an anti-TROP2 antibody.

The anti-TROP2 antibody is preferably an antibody comprising a heavy chain comprising CDRH1 consisting of an amino acid sequence represented by SEQ ID NO: 3 [=an amino acid sequence consisting of amino acid residues 50 to 54 of SEQ ID NO: 1], CDRH2 consisting of an amino acid sequence represented by SEQ ID NO: 4 [=an amino acid sequence consisting of amino acid residues 69 to 85 of SEQ ID NO: 1], and CDRH3 consisting of an amino acid sequence represented by SEQ ID NO: 5 [=an amino acid sequence consisting of amino acid residues 118 to 129 of SEQ ID NO: 1], and a light chain comprising CDRL1 consisting of an amino acid sequence represented by SEQ ID NO: 6 [=an amino acid sequence consisting of amino acid residues 44 to 54 of SEQ ID NO: 2], CDRL2 consisting of an amino acid sequence represented by SEQ ID NO: 7 [=an amino acid sequence consisting of amino acid residues 70 to 76 of SEQ ID NO: 2], and CDRL3 consisting of an amino acid sequence represented by SEQ ID NO: 8 [=an amino acid sequence consisting of amino acid residues 109 to 117 of SEQ ID NO: 2], more preferably an antibody comprising a heavy chain comprising a heavy chain variable region consisting of an amino acid sequence represented by SEQ ID NO: 9 [=an amino acid sequence consisting of amino acid residues 20 to 140 of SEQ ID NO: 1], and a light chain comprising a light chain variable region consisting of an amino acid sequence represented by SEQ ID NO: 10 [=an amino acid sequence consisting of amino acid residues 21 to 129 of SEQ ID NO: 2], and even more preferably an antibody comprising a heavy chain consisting of an amino acid sequence represented by SEQ ID NO: 12 [=an amino acid sequence consisting of amino acid residues 20 to 470 of SEQ ID NO: 1] and a light chain consisting of an amino acid sequence represented by SEQ ID NO: 13 [=amino acid residues 21 to 234 of SEQ ID NO: 2], or an antibody comprising a heavy chain consisting of an amino acid sequence represented by SEQ ID NO: 11 [=an amino acid sequence consisting of amino acid residues 20 to 469 of SEQ ID NO: 1] and a light chain consisting of an amino acid sequence represented by SEQ ID NO: 13 [=amino acid residues 21 to 234 of SEQ ID NO: 2].

The average number of units of the drug-linker conjugated per antibody molecule in the anti-TROP2 antibody-drug conjugate is preferably 2 to 8, more preferably 3 to 5, even more preferably 3.5 to 4.5, and even more preferably about 4.

The anti-TROP2 antibody-drug conjugate can be produced with reference to descriptions in WO 2015/098099 and WO 2017/002776.

In preferred embodiments, the anti-TROP2 antibody-drug conjugate is datopotamab deruxtecan (DS-1062).

### V. Therapeutic Use of the Anti-TROP2 ADC

The antibody-drug conjugate of the present disclosure can be used for treating cancer, and can be preferably used for treating at least one cancer selected from the group consisting of breast cancer (including triple negative breast cancer and hormone receptor (HR)-positive, HER2-negative breast cancer), lung cancer (including small cell lung cancer and non-small cell lung cancer), colorectal cancer (also called colon and rectal cancer, and including colon cancer and rectal cancer), gastric cancer (also called gastric adenocarcinoma), esophageal cancer, head-and-neck cancer (including salivary gland cancer and pharyngeal cancer), esophagogastric junction adenocarcinoma, biliary tract cancer (including bile duct cancer), Paget's disease, pancreatic cancer, ovarian cancer, uterine carcinosarcoma, urothelial cancer, prostate cancer, bladder cancer, gastrointestinal stromal tumor, uterine cervix cancer, squamous cell carcinoma, peritoneal cancer, liver cancer, hepatocellular cancer, corpus uteri carcinoma, kidney cancer, vulval cancer, thyroid cancer, penis cancer, leukemia, malignant lymphoma, plasmacytoma, myeloma, glioblastoma multiforme, osteosarcoma, sarcoma, and melanoma, and can be more preferably used for treating at least one cancer selected from the group consisting of breast cancer (preferably triple negative breast cancer and hormone receptor (HR)-positive, HER2-negative breast cancer), lung cancer (preferably non-small cell lung cancer, including non-small cell lung cancer with actionable genomic alterations and non-small cell lung cancer without actionable genomic alterations, wherein the actionable genomic alterations include EGFR, ALK, ROS1, NTRK, BRAF, RET, and MET exon 14 skipping), colorectal cancer, gastric cancer, pancreatic cancer, ovarian cancer, prostate cancer, and kidney cancer. Furthermore, the antibody-drug conjugate of the present disclosure can preferably be used for treating cancer that is deficient in Homologous Recombination (HR) dependent DNA DSB repair activity, or cancer that is not deficient in Homologous Recombination (HR) dependent DNA DSB repair activity.

The antibody-drug conjugate of the present disclosure can be preferably used for mammals, and can be more preferably used for humans.

The antitumor effect of the antibody-drug conjugate of the present disclosure can be confirmed by, for example, generating a model in which cancer cells are transplanted to a test animal, and measuring reduction in tumor volume, life-prolonging effects due to applying the antibody-drug conjugate of the present disclosure.

In addition, the antitumor effect of the antibody-drug conjugate of the present disclosure can be confirmed, in a clinical study, with the Response Evaluation Criteria in Solid Tumors (RECIST) evaluation method, WHO's evaluation method, Macdonald's evaluation method, measurement of body weight, and other methods; and can be determined by indicators such as Complete response (CR), Partial response (PR), Progressive disease (PD), Objective response rate (ORR), Duration of response (DoR), Progression-free survival (PFS), and Overall survival (OS).

The antibody-drug conjugate of the present disclosure can retard growth of cancer cells, suppress their proliferation, and further can kill cancer cells. These effects can allow cancer patients to be free from symptoms caused by cancer or can achieve an improvement in the QOL of cancer patients and attain a therapeutic effect by sustaining the lives of the cancer patients. Even if the antibody-drug conjugate does not accomplish the killing of cancer cells, it can achieve higher quality of life (QOL) of cancer patients while achieving longer-term survival, by inhibiting or controlling the growth of cancer cells.

The antibody-drug conjugate of the present invention can be expected to exert a therapeutic effect by application as systemic therapy to patients, and additionally, by local application to cancer tissues.

The antibody-drug conjugate of the present disclosure, in another aspect, provides for use as an adjunct in cancer therapy with ionizing radiation or other chemotherapeutic agents. For example, in the treatment of cancer, the treatment may comprise administering to a subject in need of treatment a therapeutically-effective amount of the antibody-drug conjugate, simultaneously or sequentially with ionizing radiation or other chemotherapeutic agents.

The antibody-drug conjugate of the present disclosure can be used as adjuvant chemotherapy combined with surgery operation. The antibody-drug conjugate of the present disclosure may be administered for the purpose of reducing tumor size before surgical operation (referred to as preoperative adjuvant chemotherapy or neoadjuvant therapy), or may be administered for the purpose of preventing recurrence of tumor after surgical operation (referred to as postoperative adjuvant chemotherapy or adjuvant therapy).

In further aspects, the antibody-drug conjugate of the present disclosure may be used for the treatment of cancer which is deficient in Homologous Recombination (HR) dependent DNA DSB repair activity. The HR dependent DNA DSB repair pathway repairs double-strand breaks (DSBs) in DNA via homologous mechanisms to reform a continuous DNA helix (K. K. Khanna and S. P. Jackson, Nat. Genet. 27 (3): 247-254 (2001)). The components of the HR dependent DNA DSB repair pathway include, but are not limited to, ATM (NM_000051), RAD51 (NM_002875), RAD51L1 (NM_002877), RAD51C (NM_002876), RAD51L3 (NM_002878), DMC1 (NM_007068), XRCC2 (NM_005431), XRCC3 (NM_005432), RAD52 (NM_002879), RAD54L (NM_003579), RAD54B (NM_012415), BRCA1 (NM_007295), BRCA2 (NM_000059), RAD50 (NM_005732), MRE11A (NM_005590) and NBS1 (NM_002485). Other proteins involved in the HR dependent DNA DSB repair pathway include regulatory factors such as EMSY (Hughes-Davies, et al., Cell, 115, pp 523-535). HR components are also described in Wood, et al., Science, 291, 1284-1289 (2001). A cancer that is deficient in HR dependent DNA DSB repair may comprise or consist of one or more cancer cells which have a reduced or abrogated ability to repair DNA DSBs through that pathway, relative to normal cells, i.e., the activity of the HR dependent DNA DSB repair pathway may be reduced or abolished in the one or more cancer cells. The activity of one or more components of the HR dependent DNA DSB repair pathway may be abolished in the one or more cancer cells of an individual having a cancer which is deficient in HR dependent DNA DSB repair. Components of the HR dependent DNA DSB repair pathway are well characterised in the art (see for example, Wood, et al., Science, 291, 1284-1289 (2001)) and include the components listed above.

In some embodiments, the cancer cells may have a BRCA1 and/or a BRCA2 deficient phenotype, i.e., BRCA1 and/or BRCA2 activity is reduced or abolished in the cancer cells. Cancer cells with this phenotype may be deficient in BRCA1 and/or BRCA2, i.e., expression and/or activity of BRCA1 and/or BRCA2 may be reduced or abolished in the cancer cells, for example by means of mutation or polymorphism in the encoding nucleic acid, or by means of amplification, mutation or polymorphism in a gene encoding a regulatory factor, for example the EMSY gene which encodes a BRCA2 regulatory factor (Hughes-Davies, et al., Cell, 115, 523-535). BRCA1 and BRCA2 are known tumor suppressors whose wild-type alleles are frequently lost in tumors of heterozygous carriers (Jasin M., Oncogene, 21 (58), 8981-93 (2002); Tutt, et al., Trends Mol Med., 8 (12), 571-6, (2002)). The association of BRCA1 and/or BRCA2 mutations with breast cancer is well-characterised in the art (Radice, P. J., Exp Clin Cancer Res., 21 (3 Suppl), 9-12 (2002)). Amplification of the EMSY gene, which encodes a BRCA2 binding factor, is also known to be associated with breast and ovarian cancer. Carriers of mutations in BRCA1 and/or BRCA2 are also at elevated risk of certain cancers, including breast, ovary, pancreas, prostate, hematological, gastrointestinal and lung cancer. In some embodiments, the individual is heterozygous for one or more variations, such as mutations and polymorphisms, in BRCA1 and/or BRCA2 or a regulator thereof. The detection of variation in BRCA1 and BRCA2 is well-known in the art and is described, for example in EP 699 754, EP 705 903, Neuhausen, S. L. and Ostrander, E. A., Genet. Test, 1, 75-83 (1992); Chappnis, P. O. and Foulkes, W. O., Cancer Treat Res, 107, 29-59 (2002); Janatova M., et al., Neoplasma, 50 (4), 246-505 (2003); Jancarkova, N., Ceska Gynekol., 68{1), 11-6 (2003)). Determination of amplification of the BRCA2 binding factor EMSY is described in Hughes-Davies, et al., Cell, 115, 523-535).

Mutations and polymorphisms associated with cancer may be detected at the nucleic acid level by detecting the presence of a variant nucleic acid sequence or at the protein level by detecting the presence of a variant (i.e. a mutant or allelic variant) polypeptide.

The antibody-drug conjugate of the present disclosure may be administered as a pharmaceutical composition containing at least one pharmaceutically suitable ingredient. The pharmaceutically suitable ingredient can be suitably selected and applied from formulation additives or the like that are generally used in the art, in accordance with the dosage, administration concentration or the like of the antibody-drug conjugate used in the present disclosure. For example, the antibody-drug conjugate used in the present disclosure can be administered as a pharmaceutical composition containing a buffer such as a histidine buffer, an excipient such as sucrose or trehalose, and a surfactant such as Polysorbate 80 or 20. The pharmaceutical product containing the antibody-drug conjugate used in the present disclosure can be preferably used as an injection, can be more preferably used as an aqueous injection or a lyophilized injection, and can be even more preferably used as a lyophilized injection.

In the case that the pharmaceutical product containing the anti-TROP2 antibody-drug conjugate used in the present disclosure is an aqueous injection, it can be preferably diluted with a suitable diluent and then given as an intravenous infusion. For the diluent, a dextrose solution, physiological saline, and the like, can be exemplified, and a dextrose solution can be preferably exemplified, and a 5% dextrose solution can be more preferably exemplified. In the case that the pharmaceutical product of the present disclosure is a lyophilized injection, it can be preferably dissolved in water for injection, subsequently a required amount can be diluted with a suitable diluent and then given as an intravenous infusion. For the diluent, a dextrose solution, physiological saline, and the like, can be exemplified, and a dextrose solution can be preferably exemplified, and a 5% dextrose solution can be more preferably exemplified.

Examples of the administration route which may be used to administer the pharmaceutical product of the present disclosure include intravenous, intradermal, subcutaneous, intramuscular, and intraperitoneal routes, and preferably include an intravenous route.

The anti-TROP2 antibody-drug conjugate used in the present disclosure can be administered to a human once at intervals of 1 to 180 days, and can be preferably administered once a week, once every 2 weeks, once every 3 weeks, or once every 4 weeks, and can be even more preferably administered once every 3 weeks. Also, the antibody-drug conjugate used in the present invention can be administered at a dose of about 0.001 to 100 mg/kg, and can be preferably administered at a dose of 0.8 to 12.4 mg/kg. For example, the anti-TROP2 antibody-drug conjugate can be administered once every 3 weeks at a dose of 0.27 mg/kg, 0.5 mg/kg, 1.0 mg/kg, 2.0 mg/kg, 4.0 mg/kg, 6.0 mg/kg, or 8.0 mg/kg, and can be preferably administered once every 3 weeks at a dose of 6.0 mg/kg.

### VI. Method of Predicting a Patient Response to an Anti-TROP2 Antibody-Drug Conjugate

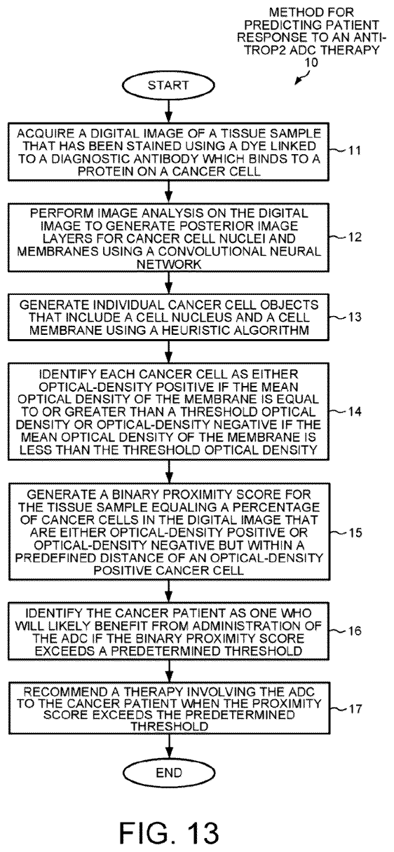

FIG. 13 is a flowchart of steps 11-17 of a method 10 by which an analysis system analyzes a digital image of tissue from a cancer patient and predicts how the cancer patient will likely respond to a therapy involving an anti-TROP2 antibody-drug conjugate (ADC). In one embodiment, the ADC is datopotamab deruxtecan (DS-1062). In one embodiment, the method predicts the response to an ADC of a patient having a cancer selected from the group consisting of breast cancer, gastric cancer, colorectal cancer, lung cancer, esophageal cancer, head-and-neck cancer, esophagogastric junction cancer, biliary tract cancer, Paget's disease, pancreatic cancer, ovarian cancer, uterine cancer sarcoma, bladder cancer, prostate cancer, urothelial cancer, gastrointestinal stromal tumor, uterine cervix cancer, squamous cell carcinoma, peritoneal cancer, liver cancer, hepatocellular cancer, endometrial cancer, kidney cancer, vulval cancer, thyroid cancer, penis cancer, leukemia, malignant lymphoma, plasmacytoma, myeloma, glioblastoma multiforme, sarcoma, osteosarcoma, and melanoma. In one embodiment, the method predicts the response to an ADC of patient having a cancer selected from the group consisting of breast cancer, gastric cancer, colorectal cancer, non-small cell lung cancer, esophageal cancer, head-and-neck cancer, esophagogastric junction adenocarcinoma, biliary tract cancer, Paget's disease, pancreatic cancer, ovarian cancer, uterine carcinosarcoma, bladder cancer, and prostate cancer. In one embodiment, the method predicts the response to an ADC of a patient with breast cancer. In another embodiment, the method predicts the ADC response of a patient with gastric cancer. In yet another embodiment, the method predicts the ADC response of a patient with lung cancer.

In a first step 11, a high-resolution digital image is acquired of a tissue slice from the cancer patient that has been stained using one or more biomarkers or stains. To predict the efficacy of the ADC therapy, a diagnostic antibody (e.g., a diagnostic biomarker) with an attached dye is used that targets the same protein as that targeted by the ADC therapy. In one embodiment, the anti-TROP2 ADC therapy to which the scoring is directed is an anti-TROP2 antibody conjugated to a drug-linker via a thioether bond, wherein the drug-linker is represented by the formula:

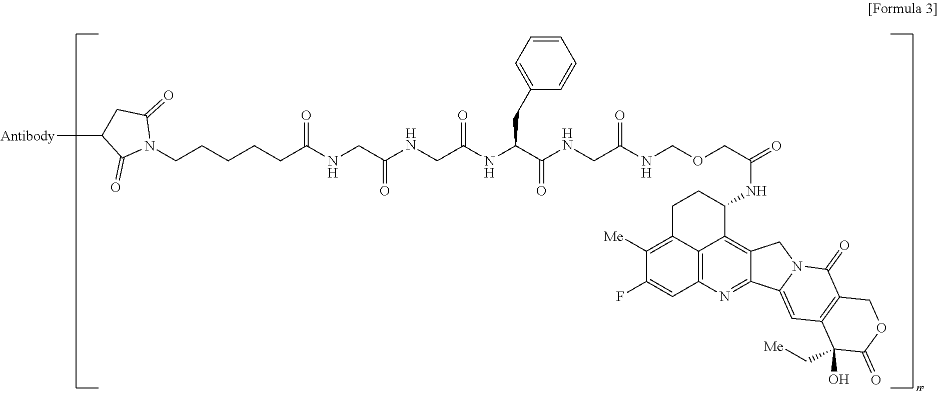

wherein “Antibody-” represents the position at which the anti-TROP2 antibody is connected. In one embodiment, the anti-TROP2 ADC to which the scoring is directed is datopotamab deruxtecan (DS-1062). Thus, in various embodiments, the diagnostic biomarker also targets the TROP2 protein.

In step 12, a pretrained convolutional neural network processes a digital image of tissue of the cancer patient that has been stained with the diagnostic antibody linked to the dye, such as 3,3′-Diaminobenzidine (DAB). The staining intensity of the dye in the membrane of a cancer cell is determined based on the mean staining intensity of the dye of all pixels associated with the corresponding segmented membrane object. Moreover, the staining intensity of the dye in a single pixel is computed based on the red, green and blue color components of the pixel. The result of the image analysis processing is two posterior image layers representing, for each pixel in the digital image, the probability that the pixel belongs to a cell nucleus and the probability that the pixel belongs to a cell membrane.

In step 13, individual cancer cells are detected based on a heuristic image analysis of the posterior layers for nuclei and membranes. Cancer cell objects are generated that include cell membrane objects.

In step 14, each cancer cell is identified as being either optical-density positive or optical-density negative based on the amount of DAB in the cell membrane. The amount of DAB is determined by the staining intensity of each membrane based on the mean (average) optical density of the brown diaminobenzidine (DAB) signal in all of the pixels of the membrane. Each cancer cell is identified as either (i) optical-density positive if the mean optical density of the cancer cell is equal to or greater than an optical density threshold, or (ii) optical-density negative if the mean optical density of the cancer cell is less than the optical density threshold. In one embodiment, the optical density threshold is in a range of 23 to 27 on a scale with a maximum optical density of 255. For example, the optical density threshold is 25. The maximum optical density that can be represented by 8 bits of RGB data is 255. The maximum optical density observed in the DAB staining of membranes was about 220.

In step 15, a binary proximity score for the digital image of the tissue sample is generated equaling the percentage of cancer cells in the digital image that are either optical-density positive or optical-density negative but within a predefined distance of an optical-density positive cancer cell. In one embodiment, the predefined distance is fifty microns. In another embodiment, the predefined distance is twenty-five microns.

In step 16, the cancer patient is identified as one who will likely benefit from administration of the anti-TROP2 ADC if the proximity score exceeds a predetermined percentage threshold. In one embodiment, the predetermined percentage threshold is in a range of 95% to 100%. For example, the predetermined percentage threshold is 99.975%. In some embodiments, a patient is designated as QCS Positive if at least 95% of the cancer cells in the digital image are either optical-density positive or optical-density negative but disposed within the predefined distance of an optical-density positive cancer cell, i.e., if the proximity score is at least 95%. In some embodiments, a patient is QCS Positive if the proximity score is at least 98%. In some embodiments, a patient is QCS Positive if the proximity score is at least 99%. In some embodiments, a patient is QCS Positive if the proximity score is at least 99.9%.

The threshold optical density used in step 14, the predefined distance used in step 15, and the predetermined percentage threshold used in step 16 are optimized using a training cohort of patients with known responses to the ADC therapy. Optimization is performed by finding the lowest log-rank p value using Kaplan-Meier analysis to stratify patients into groups with tumor shrinkage and tumor growth. Progression free survival (PFS) is mapped onto Kaplan-Meier curves, although the PFS does not relate to the time of survival of the patients but rather to the time over which there is no tumor growth.

Thus, the binary proximity score is indicative of how the cancer patient will respond in terms of tumor growth to a therapy involving an anti-TROP ADC.

In step 17, the therapy involving the anti-TROP2 ADC is recommended to score-positive patients if the score exceeds the predetermined percentage threshold.

### VII. Examples of Prediction and Scoring Method

### A. Image Analysis of Stained Tissue

The method of FIG. 13 is now described in relation to a particular image of stained cancer tissue.

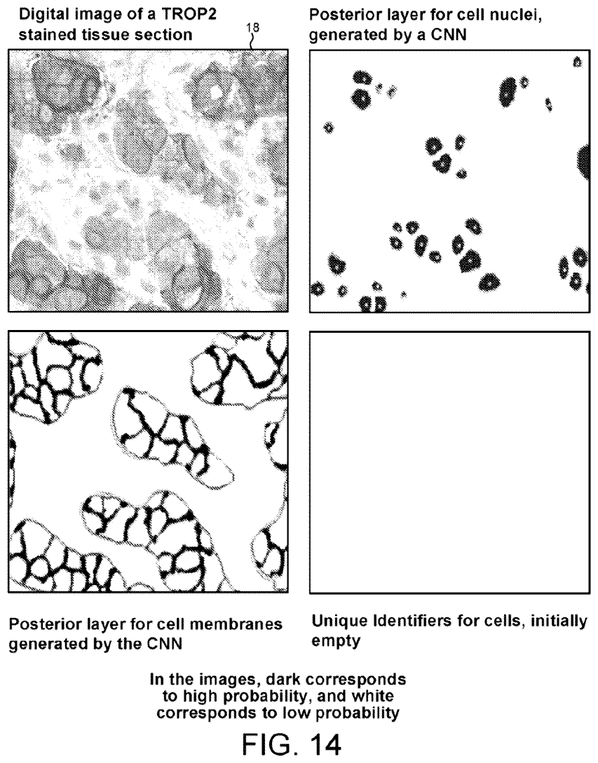

In step 11, a tissue sample is immunohistochemically stained using a dye linked to a diagnostic antibody that binds to the associated protein on the cancer cells in the tissue sample. FIG. 14 (upper-left image) is a digital image 18 of a portion of stained tissue that was acquired in step 11. Image 18 shows tissue from a cancer patient that has been immunohistochemically stained with an anti-TROP2 diagnostic antibody linked to a dye. In this example, the diagnostic antibody is an anti-TROP2 generated clone reformatted as a rabbit anti-human TROP2 IgG1 clone. IgG1 indicates the isotype of the anti-TROP2 antibody. The anti-TROP2 antibody binds to the transmembrane protein TROP2 so that the 3,3′-Diaminobenzidine (DAB) stain indicates the location of the protein TROP2 in the tissue sample.

In step 12, image analysis is performed on the digital image 18 to generate posterior image layers of cancer cell nuclei and membranes using a convolutional neural network. The image analysis is used to detect the cancer cells and their components, such as the nuclei, the membrane and the cytoplasm. FIG. 14 illustrates the image analysis process of step 12. The convolutional neural network generates posterior layers (gray value images) that indicate, for each pixel of digital image 18, the probability that each pixel belongs to either the nucleus (FIG. 14 upper-right image) or the membrane (FIG. 14 lower-left image) of the cell. High probabilities are shown in black, low probabilities in white. The upper-right image also shows the nuclei centers that are first identified in order to generate the cell membrane objects by growing outwards from the nuclei centers until meeting growth from another nucleus center.

In one embodiment, the convolutional neural network includes a series of convolution layers from the input image 18 towards a bottleneck layer with very low spatial size (1 to 16 pixels), and a series of deconvolution layers towards the posterior layers that have the same size as the input image 18. This network architecture is called a U-Net. The training of the weights of the convolutional neural networks is performed by generating manual annotation layers for nuclei and membranes in multiple training images, and then adjusting by an optimization algorithm the network weights so that the generated posterior layers are most similar to the manually generated annotation layers.

In another embodiment, the annotation layers for nuclei and membranes are generated automatically and corrected manually in multiple training images. Epithelium regions and nuclei centers are manually annotated as regions and points, respectively. For each training image, the membrane segmentation is automatically generated by applying a region growing-like algorithm (e.g., watershed segmentation) seeded by the annotated nuclei centers and constrained by the extent of the annotated epithelium region. Given a training image, the nuclei segmentation is automatically generated by applying a blob detection algorithm (e.g., by the maximally-stable-extremal-regions MSER algorithm) and by selecting as nuclei only the detected blobs that contain an annotated nucleus center. The automatically generated membrane and nuclei segmentations are visually reviewed and manually corrected if necessary. The correction steps involve one of the following methods: rejecting incorrectly segmented membranes or nuclei, explicitly accepting correctly annotated membranes or nuclei, or refining the shapes of the membranes or nuclei. For each image with annotated membranes or nuclei, an annotation layer is created. In one embodiment, each pixel of the annotation layer is assigned a “1” if it belongs to the annotated object (membrane or nucleus); otherwise it is assigned a “0”. In another embodiment, the pixels of the annotation layer represent the distance to the nearest annotated object. The network weights are adjusted by an optimization algorithm so that the generated posterior layers are most similar to the automatically generated membrane and nuclei annotation layers.

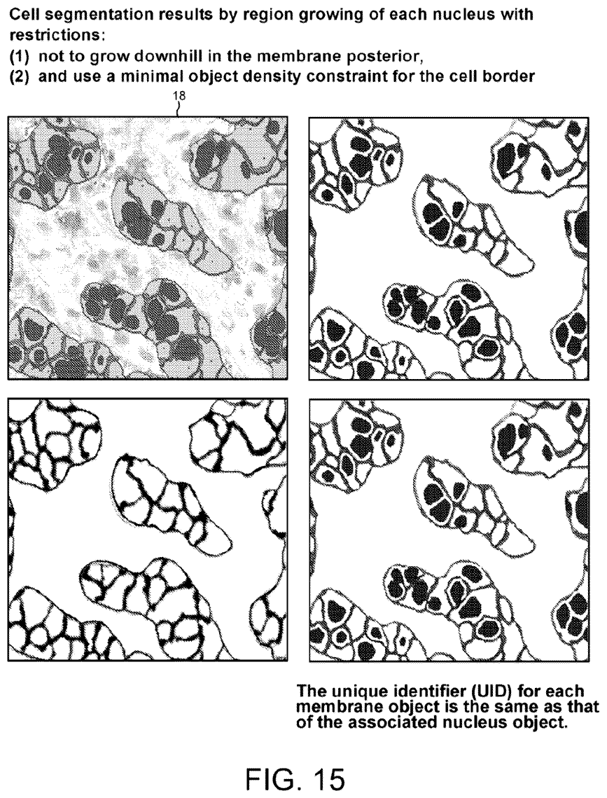

FIG. 15 illustrates step 13 in which individual cancer cell objects are detected that each include a cell membrane and a cell cytoplasm. A heuristic image analysis process uses watershed segmentation to segment the cell nuclei using the nucleus posterior layer generated by the convolutional neural network. The segmentation generates nucleus objects. Each nucleus object is assigned a unique identifier (UID). The individually identified nuclei are shown as dark objects in FIG. 15 (lower-right image). The detected nuclei are also displayed as overlays in the input image 18 (upper-left image) and in the posterior layers for nuclei (upper-right image) and membranes (lower-left image).

In one embodiment, the watershed segmentation involves a thresholding of the nucleus posterior layer with a predefined first size threshold. All single connected pixels that are above a first size threshold are considered to belong to a nucleus object. Nucleus objects with an area smaller than 16 μm\hat{ }2 are discarded. A UID is assigned to each nucleus object. In a subsequent step, the nucleus objects are grown towards smaller nucleus posteriors in which the added nucleus posterior pixels must be greater than a second predefined threshold.

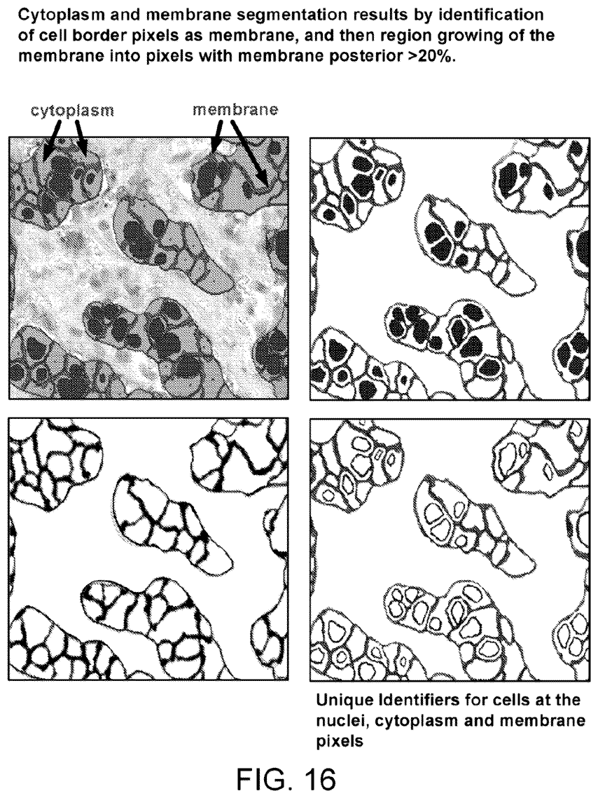

FIG. 16 illustrates the detection and segmentation of membrane objects which are segmented by growing the region of the border pixels of detected cells outwards to the membrane probability layer and to a predefined membrane layer posterior threshold value. The thicker border regions become the membrane objects. Each membrane object is assigned the same UID as that of the associated nucleus object.

The space between the membrane and the nucleus is assigned to the cytoplasm using the UID of the nucleus. For each membrane (FIG. 16 see top-left image) and cytoplasm (FIG. 16 see top-left image), the average optical densities of the DAB staining is exported to a file on a hard drive together with the UIDs. For each cell (defined as including a nucleus, cytoplasm and membrane), the position of the center of gravity (x,y) of the cell within the slide is also exported. The file may reside on a hard disk, a solid state disk or a portion of dedicated RAM in a computer system.

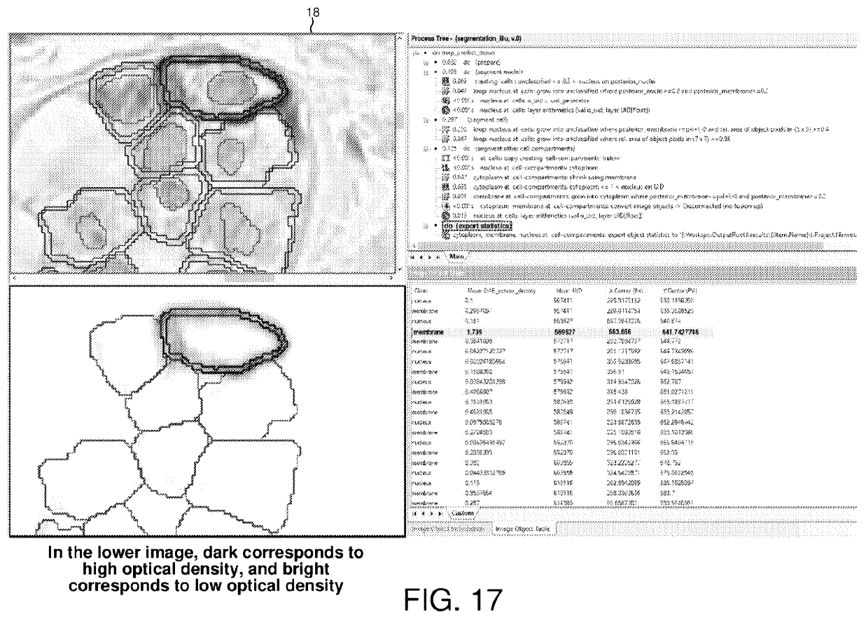

FIG. 17 illustrates the results of the image analysis in an image analysis software environment. FIG. 17 (upper-left image) shows the segmentation of nucleus objects and membrane objects as an overlay on a digital image of stained tissue. FIG. 17 (lower-left image) shows the segmentation of a nucleus object as an overlay on an optical density representation of the digital image. Dark optical density pixels are associated with a high amount of DAB, and bright optical density pixels are associated with a low amount of DAB. The DAB optical density of each image pixel is computed from the red-green-blue representation of the image pixel by transformation of the red-green-blue color space so that the brown DAB component becomes an independent color, and by taking the logarithm of that brown color component. FIG. 17 (upper-right image) shows the image analysis script used to generate the segmented image. FIG. 17 (lower-right image) shows the exported measurements for all cell membrane objects and cytoplasm objects in image 18.

### B. Calculation of a Predictive Efficacy Score

Based on the optical density of the DAB staining within the membrane objects and optionally the cytoplasm objects, a spatial proximity score is determined for the tissue sample shown in the digital image 18. The spatial proximity score also accounts for the staining intensities of the DAB dye in the membrane objects and optionally cytoplasm objects of neighboring cancer cells that are closer than a predefined distance to the cancer cell for which the single-cell ADC score is being computed. There are two types of spatial proximity scores: a binary spatial proximity score (bSPS) and a continuous spatial proximity score (cSPS). The spatial proximity scores estimate the effect of the bystander activity of the ADC drug. Bystander activity is characterized by local toxicity of ADC payloads released from cells that internalize the ADC drug. The effective range of the local toxicity is represented as the predefined distance parameter in the binary and continuous spatial proximity scores.

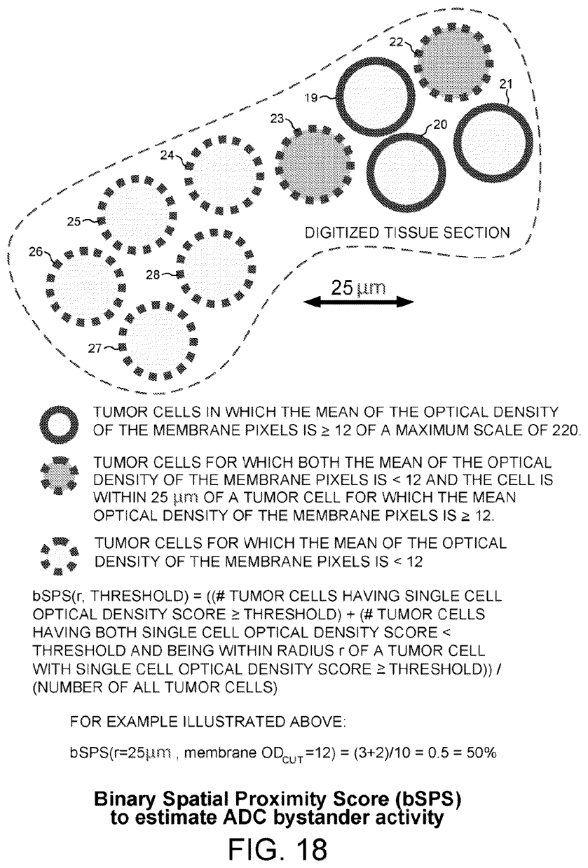

FIG. 18 shows an example of how the binary spatial proximity score (bSPS) is determined. The binary spatial proximity score is a percentage score equal to the sum of (i) the number of cancer cells in the digital image 18 whose staining intensity equals or exceeds an optical density threshold plus (ii) the number of cancer cells in the digital image whose staining intensity is less than the optical density threshold but that are located within a predefined distance of at least one cancer cell whose staining intensity equals or exceeds the optical density threshold, the sum divided by the total number of cancer cells in the digital image 18.

FIG. 18 illustrates ten exemplary cancer cells (also called tumor cells) with various optical densities of staining from the image analysis of steps 12-13. The steps of heuristic image analysis illustrated in FIGS. 14-16 are used to obtain the segmentation into image objects including cancer cell objects, cell membrane objects and cytoplasm objects. Circles with solid lines around the circumference represent tumor cells whose optical density of brown DAB staining (corresponding to the amount of target protein TROP2) is greater than or equal to a predetermined optical density threshold. In this example, the optical density threshold is twelve from a maximum scale of optical density of 255. Three cancer cells 19-21 in FIG. 18 are classified as optical-density positive. Circles with dashed circumferences represent tumor cells whose optical density of brown DAB staining is less than the predetermined optical density threshold. Seven cancer cells 22-28 are classified as optical-density negative. Darker gray circles with dashed circumferences represent optical-density negative cancer cells that are located within a predefined distance of at least one optical-density positive cancer cell. The two cancer cells 22-23 are optical-density negative but also disposed within the predefined distance of an optical-density positive cancer cell. In this example, the predefined distance is 25 microns.

In this example, the binary spatial proximity score equals the percentage score of 50%, which is calculated as shown in FIG. 18 as the sum of three optical-density positive cells and two optical-density negative that are within the predefined distance of an optical-density positive cell, the sum divided by the total of ten cancer cells ([3+2]/10=0.5).

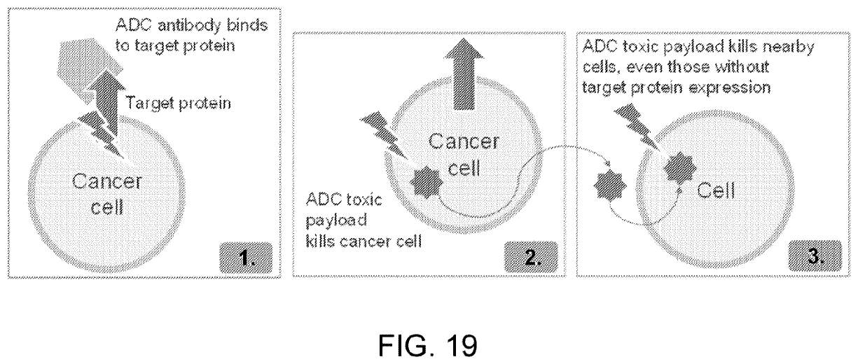

In the schematic image of FIG. 18, the three cancer cells 19-21 express a high amount of the target protein TROP2 and would very likely be killed by the ADC payload (e.g., cytotoxin) entering the cell linked to the ADC antibody (effect 1 in FIG. 19). The two cancer cells 22-23 do not express sufficient amounts of the target protein TROP2 to be killed directly by the anti-TROP2 ADC. However, due to the proximity of at least one of the optical-density positive cells 19-21, the toxic payload released from cells 19-21 would also kill the cancer cells 22-23 (effect 3 in FIG. 19). The remaining optical-density negative cancer cells 24-28 would remain active and could be the origin of a drug resistance mechanism, which could eventually cause the death of the patient.

FIG. 19 illustrates the mechanism by which an anti-TROP2 ADC therapy kills cancer cells. In a first step, the ADC antibody binds to the target protein TROP2 and inhibits the natural function of the target protein, which may lead to cell death. In a second step, the payload (e.g., a type I topoisomerase inhibitor) is internalized into the cell and kills the cell by the toxicity of the payload. This uptake of the payload depends on the amount of target protein on the membrane, and also on the difference in the amount of target protein on the membrane and in the cytoplasm. After uptake, the payload can be released from the cell into the surrounding tissue. In a third step, the payload may enter nearby cells and may kill them as well. The spatial distribution of the payload in the tissue is spread by passive diffusion.

Traditional IHC scoring reflects both the effect of inhibition of the target protein due to ADC binding, as well as the effect of the cytotoxic payload entering a cancer cell together with the ADC antibody. Thus, the traditional scoring for ADC therapies does not reflect the importance of the presence of the target protein in the cytoplasm and the effect of the cytotoxic payload that diffuses into the tissue after being released from the first killed cancer cell. In comparison, the novel predictive spatial proximity score measures the effect of the release of the cytotoxic payload on neighboring cancer cells.

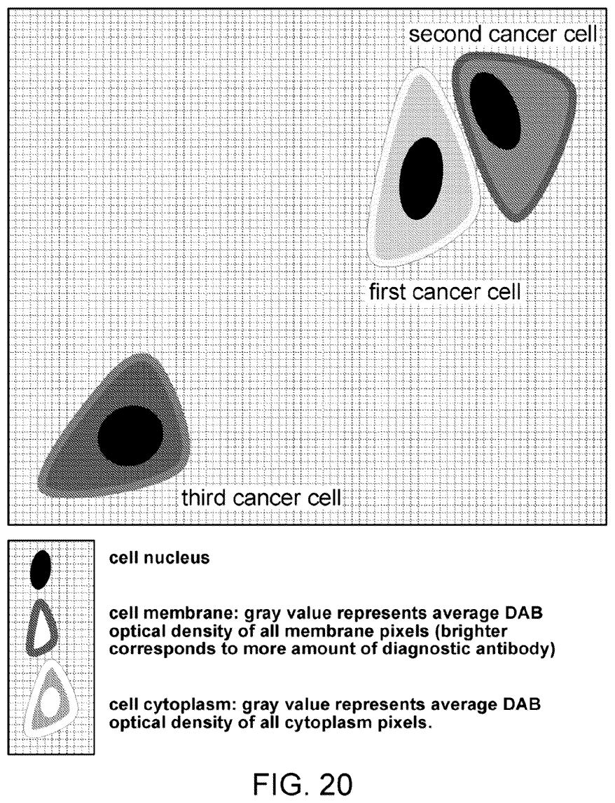

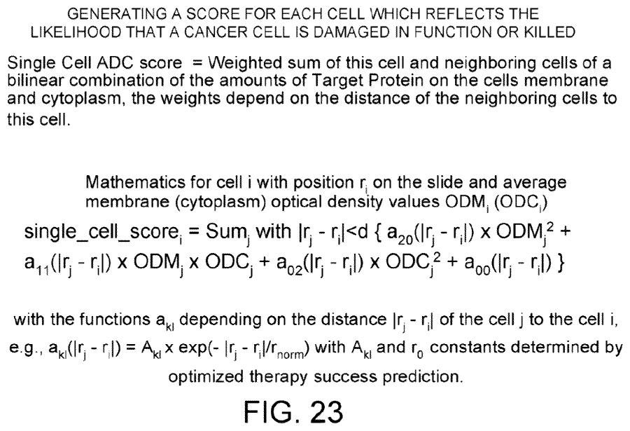

FIGS. 20-22 illustrate an example of how the continuous spatial proximity score is determined. Although the continuous spatial proximity score applies a predefined distance, optical-density negative cancer cells are still attributed some weight despite being disposed farther than the predefined distance from an optical-density positive cancer cell. The weighting of optical-density negative cancer cells disposed beyond the predefined distance can be either Gaussian or linear. The formula for the continuous spatial proximity score shown in FIG. 23 applies a Gaussian weighting. In addition, this embodiment of the continuous spatial proximity score also takes into account staining in the cytoplasm. The continuous spatial proximity score is computed for the tissue sample shown in the digital image 18 based on the optical density of the DAB staining within the membrane objects and optionally the cytoplasm objects.

FIG. 20 illustrates exemplary quantitative results of the optical density of staining from the image analysis of steps 12-13 in a schematic drawing using gray values of membrane and cytoplasm pixels. The steps of heuristic image analysis illustrated in FIGS. 14-16 are used to obtain the example segmentation of FIG. 20 into cell nuclei, cell membranes and cell cytoplasm. Bright gray values in FIG. 20 are associated with high DAB optical density, and therefore with a high amount of proteins targeted by the diagnostic antibody. Dark gray values are associated with a low DAB optical density. Brighter pixels correspond to a higher DAB optical density.

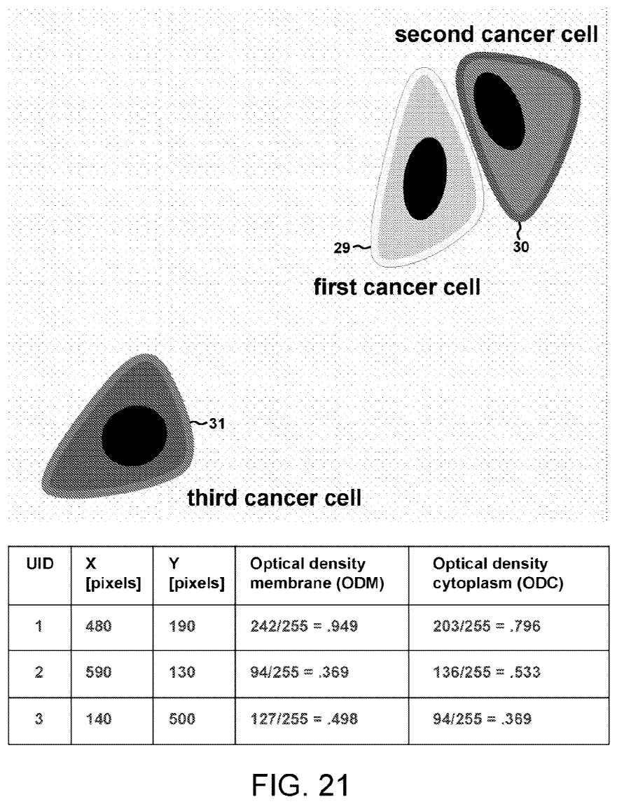

FIG. 21 lists the exemplary quantitative amounts of staining on the membranes and in the cytoplasms of the image of FIG. 20, which is reproduced in part in FIG. 21. The optical density of the brown DAB signal from the membranes of the first, second and third cells is 0.949, 0.369 and 0.498, respectively. In this example, the optical density is expressed as a percentage of the maximum staining of 255. The optical density of the brown DAB signal from the cytoplasms of the first, second and third cells is 0.796, 0.533 and 0.369, respectively. In the schematic image of FIG. 21, the first cancer cell 29 expresses a high amount of the target protein TROP2 and would be very likely to be killed by the ADC payload (e.g., cytotoxin) entering the cell linked to the ADC antibody (effect 1 in FIG. 19). The second cancer cell 30 and third cancer cell 31 do not express sufficient amounts of the target protein TROP to be killed directly by the anti-TROP2 ADC. However, due to the vicinity of the second cancer cell 30 to the first cancer cell 29, the toxic payload released from the first cancer cell would also kill the second cancer cell 30 (effect 3 in FIG. 19). The third cancer cell 31 would remain active and could be the origin of a drug resistance mechanism, which could eventually cause the death of the patient.

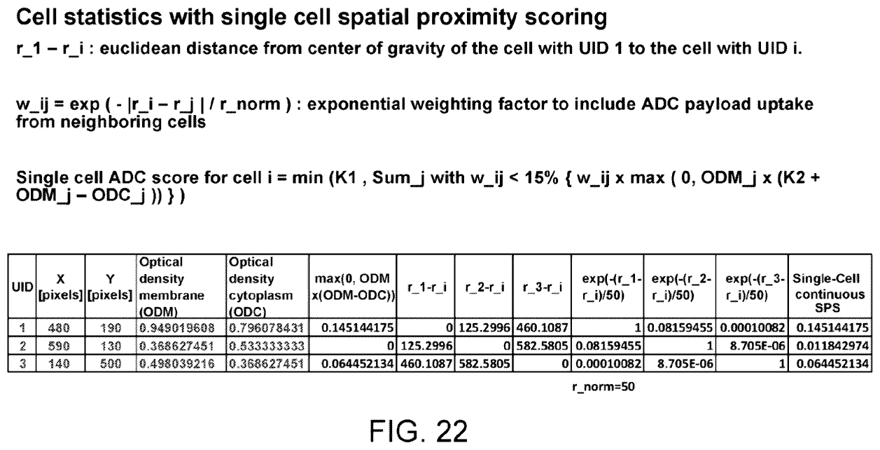

In step 14 of the method of FIG. 13, the mean optical density of each cancer cell is determined. Then in a modification of method 10 for the continuous score, a single-cell spatial proximity score is determined. FIG. 22 illustrates the calculation of the single-cell spatial proximity score for each of the three cells shown in FIG. 21 and incorporates an exponential weighting factor for cell separation to reflect the uptake of the ADC payload (e.g., cytotoxin) into neighboring cells. The single-cell score can be calculated based on the formula shown in FIG. 22. The predefined distance used in the formula is 50 microns. The optical densities listed in FIG. 21 for the DAB signal from the cell membranes (0.949, 0.369 and 0.498) and from the cytoplasms (0.796, 0.533 and 0.369) are inputs into the calculation illustrated in FIG. 22. The first, second and third cells 29, 30, 31 have single-cell spatial proximity scores of 0.145, 0.012 and 0.064, respectively.

Thus, the continuous spatial proximity score incorporates the measurement of the amount of target protein on the cell membrane using the DAB optical density and optionally an estimation of the amount of ADC payload (e.g., cytotoxin) uptake. As shown in FIG. 21, the uptake of the ADC payload (e.g., cytotoxin) for a first cell depends on both the amount of dye in its membrane and in its cytoplasm, as well as on the amount of dye in the membrane and the cytoplasm of a second cell in the vicinity of the first cell. More specifically, the vicinity may be a circular disk with a predefined radius around the first cancer cell. In one embodiment, the continuous spatial proximity score for the first cancer cell is determined by a distance-weighted sum of the several powers of DAB optical densities of membrane and cytoplasm objects whose associated cancer cell centers are closer to the first cancer cell center than a predefined distance. In one embodiment, the predefined distance is 50 μm, as used in the calculation of FIG. 22. In another embodiment, the distance is 20 μm. In yet another embodiment, the distance weighting involves computing the exponential of the scaled negative Euclidean distance from the first cancer cell center to the other cancer cell centers in the sum. In another embodiment, the powers in the sum are restricted to 0, 1, and 2 (constants, linear terms, squares).

FIG. 23 shows one embodiment of a formula for calculating the single-cell ADC score in the form of a continuous spatial proximity score. The functions aₖₗ in the formula depend on the distance |rⱼ−rᵢ| from the cell j to the cell i. ODM; is the DAB optical density of the membrane of cell j, and ODCⱼ is the DAB optical density of the cytoplasm of cell j. The constants A_ij, r_norm and d are the same for all types of cancer. However, the threshold for the score to determine whether the patient is eligible for the ADC therapy is not the same for different types of cancer.

The formula in FIG. 23 for the continuous spatial proximity score applies a Gaussian weighting to optical-density negative cancer cells disposed beyond the predefined distance from the cell for which the single-cell score is being calculated. In another embodiment, a linear weighting is given to optical-density negative cancer cells disposed beyond the predefined distance. The formula for the single-cell continuous spatial proximity score that applies a linear weighting is, for the cellᵢ:

Sumⱼ with |rⱼ−rᵢ|<d{membrane ODⱼx(1−|rⱼ−rᵢ|/d)}

### C. Validation of Binary Spatial Proximity Score.

Various QCS predicted response scores were validated based on a patient trial (J101 NCT03401385), which was a Phase I clinical trial with datopotamab deruxtecan (DS-1062). The dataset of the J101 patient trial includes stained tissue images (using the Abcam antibody clone EPR 20043) and therapy response rates for patients with multiple cancer types. A first validation of the predictive response scores was based on data from 115 patients with non-small cell lung cancer (NSCLC). Each response score was trained using pathologists' annotations, and the performance of the scoring method was validated on unseen data to ensure its generalization and robustness. The response score was blindly applied to the J101 data. The optical density OD (level of brown DAB stain intensity) was computed on detected membranes to derive features for the response scores that were linked to progression free survival (PFS) prediction. The response score features were selected to maximize the time of no tumor growth (or tumor shrinkage).

The method of FIG. 13 involves a first response score feature of the novel QCS method, which is the binary spatial proximity score. The time of no tumor growth (progression free survival time) was maximized with the parameters of the membrane optical density cutoff being 25, and the predefined distance being 50 microns. Thus, the cohort of 115 NSCLC patients was used to validate the binary spatial proximity score denoted as bystander_memb(meanOD)_binary_r50_cut25.

In step 15 of method 13, the binary spatial proximity score is generated for the tissue sample based on the percentage of cancer cells in the digital image that are either optical-density (OD) positive or optical-density negative but within a predefined distance of an optical-density positive cancer cell. In this embodiment of the QCS method, the predefined distance is 25 microns. Each cancer cell is identified as optical-density positive if the mean optical density is greater than or equal to an optical density threshold, and optical-density negative if the mean optical density is less than the optical density threshold. In this embodiment, the optical density threshold is 25 on a scale with a maximum optical density of 255.

In step 15 of method 13, a cancer patient is identified as one who will likely benefit from the administration of the ADC (bSPS positive) if the binary spatial proximity score exceeds a predetermined threshold. In this embodiment, the predetermined threshold is 99.97%. The predefined distance, the optical density threshold and the predetermined threshold were correlated to the responses of the 115 patients in the training cohort.

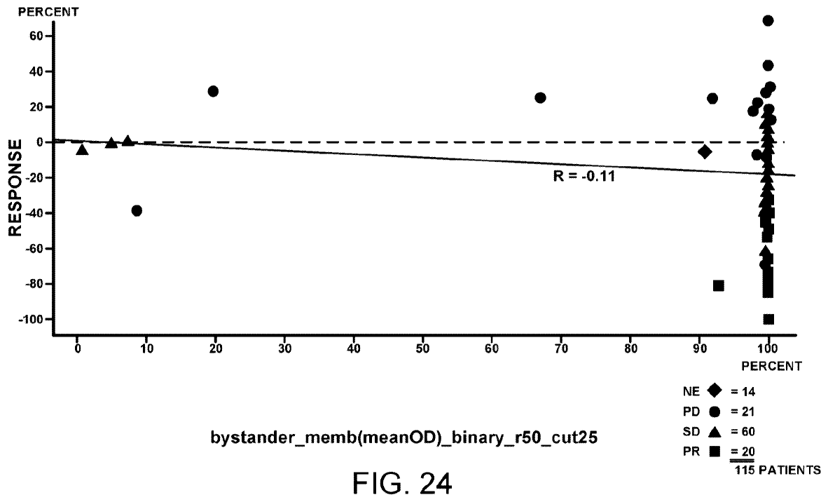

FIG. 24 illustrates how the predefined distance, the optical density threshold and the predetermined threshold were correlated to responses of tissue samples of cancer patients treated with the anti-TROP2 ADC. FIG. 24 is a scatter plot showing the correlation between actual outcomes of the 115 NSCLC patients in the J101 trial. The actual responses of the patients are denoted as progressive disease PD (circle), stable disease SD (triangle), partial response PR (square), and non-evaluable NE (diamond). The distribution of the 115 patients among the response groups was PD=21, SD=60, PR=20 and NE=14. Patients whose tumors both shrunk by less than 30% and grew by less than 30% are considered to have a stable disease. A partial response PR is defined as tumor shrinkage between 30% and 100%, and a complete response CR is tumor shrinkage of 100% and the elimination of the tumor. In the scatter plot of FIG. 24, complete responses are categorized as partial responses. Although various dosages of the ADC were administered to the 115 patients (4 mg/kg, 6 mg/kg and 8 mg/kg), no correlation of the various dosages to differing responses was observed. FIG. 24 shows the relationship between tumor growth rate in percent and the predicted response score: bystander_memb(meanOD)_binary_r50_cut25 (percentage of cancer cells that are either (i) OD positive or (ii) OD negative and within 50 microns of an OD positive cell). Y axis values greater than 0 denote that the tumor increased in size during observation, and values less than 0 signify that the tumor size shrank during observation.

The best-fit line of FIG. 24 shows that there was a negative correlation between the predicted response score bystander_memb(meanOD)_binary_r50_cut25 and the change in tumor size post treatment. The Spearman correlation rho of the best-fit line is R=−0.28. Thus, there were larger decreases in tumor size associated with higher predicted response scores.

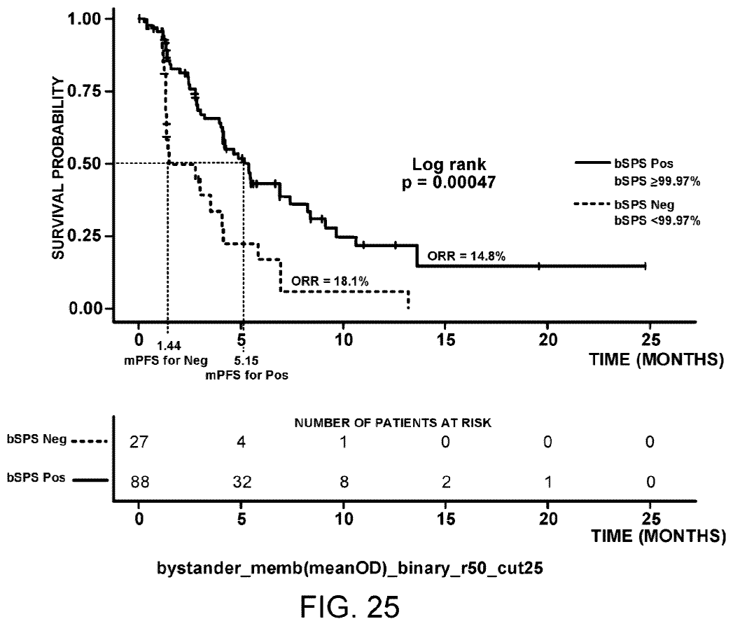

FIG. 25 is a graph of Kaplan-Meier curves of progression-free survival for two groups of the 115 NSCLC patients in the J101 trial that were stratified using the binary spatial proximity score. The method of FIG. 13 was used to divide the 115 patients into QCS Positive and QCS Negative patients. The quality of the stratification between the two Kaplan-Meier curves achieved using the QCS feature bystander_memb(meanOD)_binary_r50_cut25 is indicated by the log-rank p-value of 0.00047. There were 88 bSPS positive (QCS Positive) patients and 27 bSPS negative (QCS Negative) patients. The group of 88 bSPS positive patients exhibited an objective response rate (ORR) of 14.8% and a mean progression-free survival (mPFS) period of 5.15 months. The group of 27 bSPS negative patients exhibited an objective response rate of 18.1% and a mean progression-free survival period of 1.44 months. The upper Kaplan-Meier curve shows the group of 88 patients with better survival outcomes, which corresponds to patients for which at least 99.97% of the cells either (i) exhibited an optical density of TROP2 staining of at least 25, or (ii) were located within 50 microns of a cell exhibiting the minimal staining of 25. Thus, the scoring method of FIG. 13 identified 88 of the 151 J101 lung cancer patients as likely to benefit from an anti-TROP2 ADC therapy.

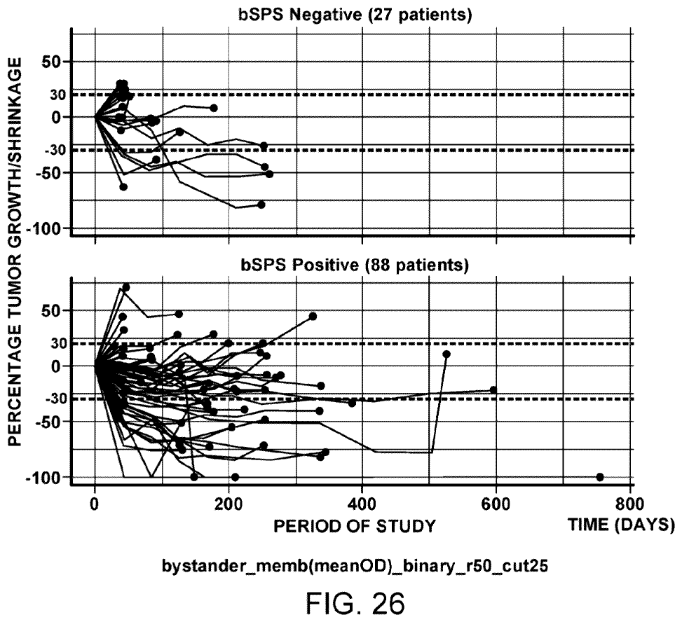

FIG. 26 shows spider plots of the 88 bSPS positive patients and the 27 bSPS negative patients of the Kaplan-Meier curves of FIG. 25. Each dot of the spider plots indicates the percentage by which the patient's tumor has shrunk or grown after the indicated period of time. The spider plots of FIG. 26 indicate time in days, whereas the progression-free survival time is shown in the Kaplan-Meier curves of FIG. 25 in months. For example, the last point at almost 25 months on the upper Kaplan-Meier curve of FIG. 25 corresponds to the lower right dot at about 750 days in the lower spider plot for bSPS positive patients. The weight of the distribution of dots in the lower spider plot for bSPS positive patients is below the 0% line, which indicates a weighting of patients whose tumors have shrunk. On the other hand, the weight of the distribution of dots in the upper spider plot for bSPS negative patients is above the 0% line, which indicates a weighting of patients whose tumors have grown.

In FIGS. 25-26, patients whose tumors have shrunk by 30% or more are categorized as having a positive response to the anti-TROP2 ADC (which includes both partial response PR and complete response CR patients). The upper spider plot for bSPS negative patients shows that only four of the 27 bSPS negative patients exhibited a positive response to the ADC. (The dot below the −30% line that had a single observation after 50 days was considered non-evaluable NE and therefore not a positive response.) In the lower spider plot for bSPS positive patients, there are 16 dots (other than NE dots) below the −30% line corresponding to patients who exhibited a positive response to the ADC.

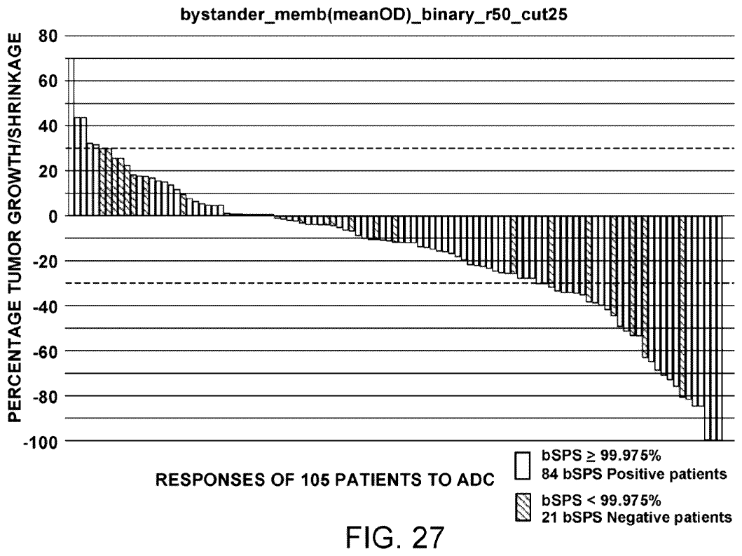

FIG. 27 is a waterfall bar graph showing the clinical response of 105 of the 115 NSCLC patients in the J101 trial ordered from greatest tumor growth to greatest tumor shrinkage. Tumor growth/shrinkage results are not shown for 10 of the non-evaluable (NE) patients. Solid bars denote bSPS Positive patients whose binary spatial proximity score equaled or exceeded 99.975%. Bars with diagonal hatching denote bSPS Negative patients whose binary spatial proximity score is less than 99.975%. The bar graph shows that the QCS predicted response score bystander_memb(meanOD)_binary r50_cut25 identifies a greater portion of the patients whose tumors shrunk after ADC treatment as bSPS Positive and a greater portion of the patients whose tumors grew after ADC treatment as bSPS Negative.

### D. Validation of Normalized Membrane OD Score

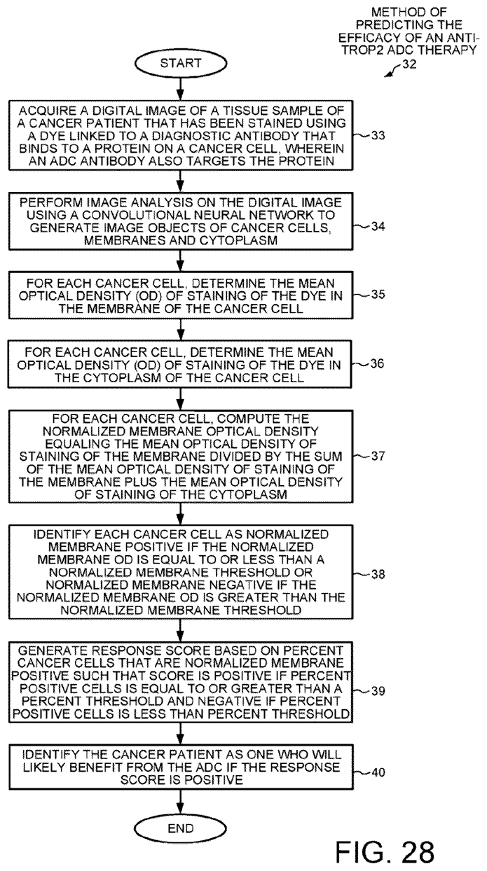

Variations of the novel method of FIG. 13 were also validated based on the patient trial (J101 NCT03401385) of 115 NSCLC patients with the anti-TROP2 ADC datopotamab deruxtecan (DS-1062). FIG. 28 is a flowchart of steps 33-40 of a method 32 of predicting the response of a cancer patient to the anti-TROP2 ADC. Method 32 is another embodiment of method 10 of FIG. 13 and predicts the efficacy of a therapy involving an anti-TROP2 ADC based on the normalized membrane optical density for each cancer cell. The normalized membrane optical density equals the mean optical density of membrane staining divided by the sum of the mean optical density of membrane staining plus the mean optical density of cytoplasm staining.

In step 33, a digital image 18 is acquired of a tissue slice from the cancer patient that has been stained using a dye linked to a diagnostic antibody that targets the same protein as that targeted by the ADC therapy, in this case the TROP2 protein. In step 34, image analysis is performed on the digital image to generate image objects of cancer cells, cell membranes and cytoplasm objects. In step 35, the mean optical density (OD) of staining of the dye in the cell membrane is determined for each cancer cell. In step 36, the mean optical density (OD) of staining of the dye in the cytoplasm is determined for each cancer cell.

In step 37, the normalized membrane optical density for each cancer cell in the digital image is computed. The normalized membrane optical density equals the mean optical density of staining of the membrane divided by the sum of the mean optical density of staining of the membrane plus the mean optical density of staining of the cytoplasm. In step 38, each cancer cell is identified as being either (i) normalized membrane positive if the normalized membrane optical density of the cancer cell is equal to or less than a normalized membrane threshold, or (ii) normalized membrane negative if the normalized membrane optical density of the cancer cell is greater than the normalized membrane threshold. In step 39, a predicted response score is generated for the tissue sample based on the percentage of cancer cells in the digital image that are identified as being normalized membrane positive. The predicted response score is positive if the percentage of cancer cells that are normalized membrane positive is equal to or greater than a percentage threshold and negative if the percentage of cancer cells that are normalized membrane positive is less than the percentage threshold. In step 40, the cancer patient is identified as one who will likely benefit from administration of the anti-TROP2 ADC if the predicted response score is positive.

The normalized membrane threshold and the percentage threshold are correlated to responses of the cohort of 115 training patients who have been treated with the TROP2 ADC. These parameters are determined by performing a progression free survival (PFS) analysis on the clinical trial data. The time of no tumor growth (progression free survival time) was maximized with the parameters of normalized membrane threshold (OD/OD) being 0.486, and the percentage threshold being 5%. Thus, the cohort of 115 NSCLC patients was used to validate the predicted response score denoted as memb(meanOD)/(memb(meanOD)+cyto(meanOD))_q5.

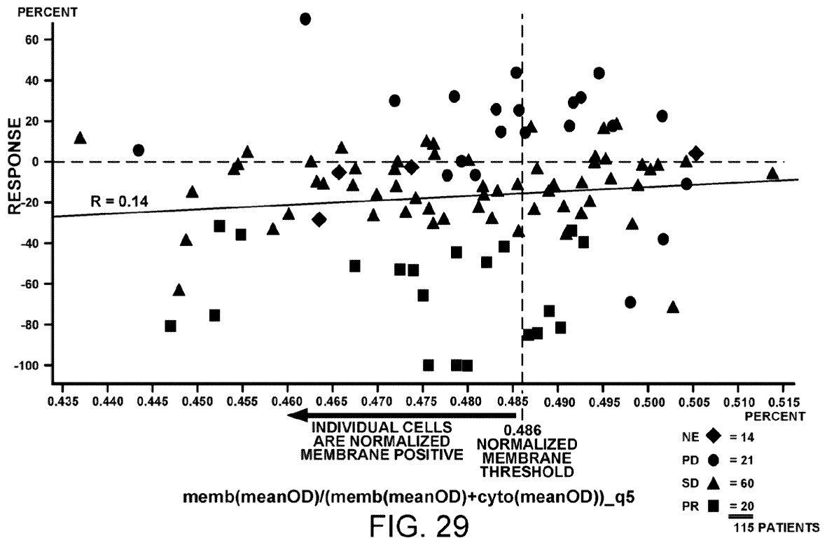

FIG. 29 illustrates how the normalized membrane threshold and the percentage threshold were correlated to responses of tissue samples of cancer patients treated with the anti-TROP2 ADC. FIG. 29 is a scatter plot showing the correlation between actual outcomes of the 115 NSCLC patients in the J101 trial. The actual responses of the patients are denoted as progressive disease PD (circle), SD stable disease SD (triangle), partial response PR (square), and non-evaluable NE (diamond). The distribution of the 115 patients among the response groups was PD=21, SD=60, PR=20 and NE=14. Patients whose tumors both shrank by less than 30% and grew by less than 30% are categorized as having a stable disease SD, whereas patients whose tumors shrank between 30% and 100% are considered to exhibit a partial response PR. In the scatter plot of FIG. 29, patients having a complete response CR due to the elimination of their tumors (100% shrinkage) are also categorized as having a partial response PR.

FIG. 29 shows the relationship between tumor growth rate in percent and the predicted response score: memb(meanOD)/(memb(meanOD)+cyto(meanOD))_q5 (normalized membrane OD). Y axis values greater than 0 signify that the tumor increased in size during observation, whereas values less than 0 indicate that the tumor size shrank during observation. The best-fit line of FIG. 29 shows that there was a positive correlation between the predicted response score memb(meanOD)/(memb(meanOD)+cyto(meanOD))_q5 and the change in tumor size post treatment. The Spearman correlation rho of the best-fit line is R=0.14. Thus, there were larger decreases in tumor size associated with lower predicted response scores.

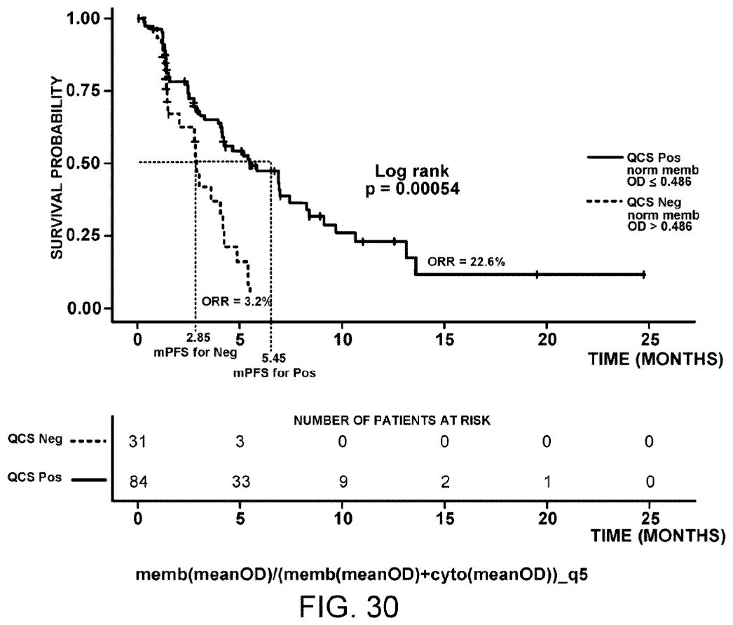

FIG. 30 is a Kaplan-Meier graph of progression-free survival that divides the 115 NSCLC patients of the J101 trial into 84 QCS Positive and 31 QCS Negative patients using method 32 of FIG. 28. The quality of the stratification between the two Kaplan-Meier curves achieved using the QCS feature memb(meanOD)/(memb(meanOD)+cyto(meanOD))_q5 is indicated by the log-rank p value of 0.00054. The group of 84 QCS Positive patients exhibited an objective response rate (ORR) of 22.6% and a mean progression-free survival (mPFS) period of 5.45 months. The group of 31 QCS Negative patients exhibited an objective response rate of 3.2% and a mean progression-free survival period of 2.85 months. The upper Kaplan-Meier curve shows the group of 84 patients with better survival outcomes, which corresponds to patients for which at least 5% of their cancer cells exhibited a normalized membrane optical density equaling or less than 0.486. Thus, the scoring method 32 of FIG. 28 identified 84 of the 151 J101 lung cancer patients as likely to benefit from an anti-TROP2 ADC therapy.

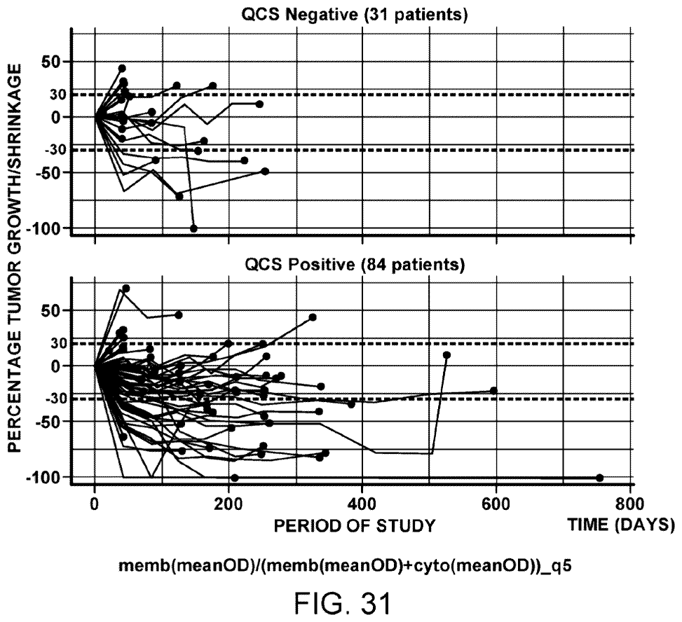

FIG. 31 shows spider plots of the 84 QCS Positive patients and the 31 QCS Negative patients of the Kaplan-Meier curves of FIG. 30. Each dot of the spider plots indicates the percentage by which the patient's tumor has shrunk or grown after the indicated period of time. The spider plots of FIG. 31 indicate time in days, whereas the progression-free survival time in the Kaplan-Meier curves of FIG. 30 is shown in months. For example, the last point at almost 25 months on the upper Kaplan-Meier curve of FIG. 30 corresponds to the lower right dot at about 750 days in the lower spider plot for QCS Positive patients. The weight of the distribution of dots in the lower spider plot for QCS Positive patients is below the line for 0% tumor growth, which indicates that method 32 has identified primarily patients whose tumors have shrunk. On the other hand, the weight of the distribution of dots in the upper spider plot for QCS Negative patients is above the 0% line, which indicates that method 32 has identified primarily patients whose tumors have grown.

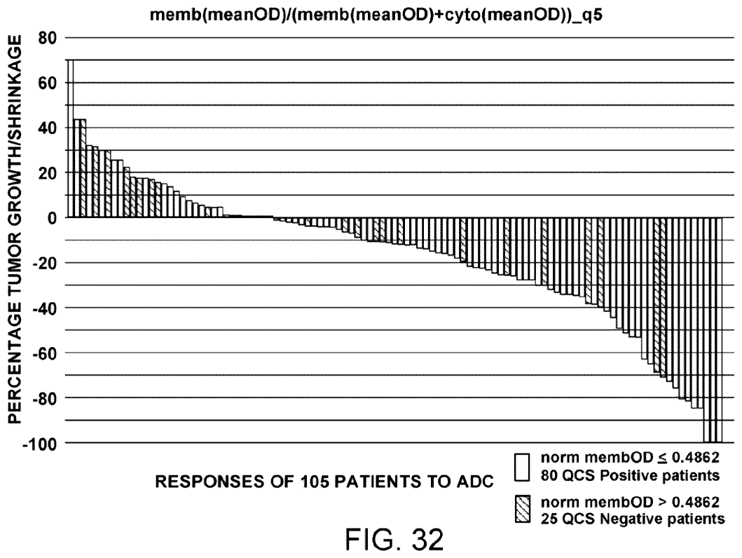

FIG. 32 is a waterfall bar graph showing the clinical response of 105 of the 115 NSCLC patients in the J101 trial ordered from greatest tumor growth to greatest tumor shrinkage. Tumor growth/shrinkage results are not shown for 10 of the non-evaluable (NE) patients. Solid bars denote QCS Positive patients who had a response score for the normalized membrane OD feature of 0.486 or below. Bars with diagonal hatching denote QCS Negative patients whose response score for the normalized membrane OD feature was above 0.486. The bar graph shows that the response score for the QCS feature memb(meanOD)/(memb(meanOD)+cyto(meanOD))_q5 identifies a greater portion of the patients whose tumors shrank after ADC treatment as QCS Positive and a greater portion of the patients whose tumors grew after ADC treatment as QCS Negative.

E. Validation of Combined bSPS and Normalized Membrane OD Score

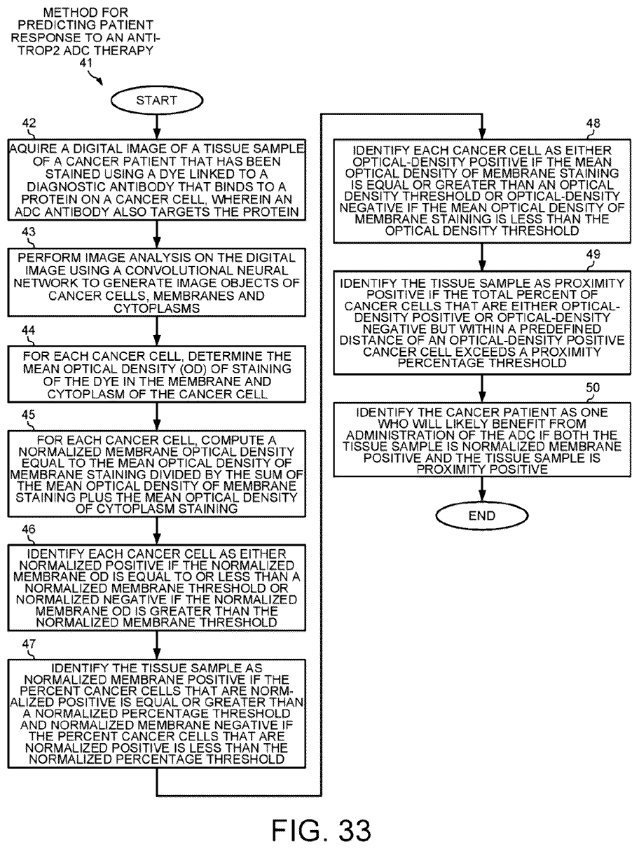

FIG. 33 is a flowchart of steps 42-50 of an additional method 41 of predicting the response of a cancer patient to the anti-TROP2 ADC. Method 41 predicts the efficacy of a therapy involving an anti-TROP2 ADC based on a combination of the binary spatial proximity score and the normalized membrane optical density.

In step 42, a digital image 18 is acquired of a tissue slice from the cancer patient that has been stained using a dye linked to a diagnostic antibody that targets the same protein as that targeted by the ADC therapy, in this case the TROP2 protein. In step 43, image analysis is performed on the digital image to generate image objects of cancer cells, cell membranes and cytoplasm objects. In step 44, the mean optical density (OD) of membrane staining and the mean OD of cytoplasm staining are determined for each cancer cell. In step 45, a normalized membrane optical density is computed for each cancer cell in the digital image 18 that is equal to the mean OD of membrane staining divided by the sum of the mean OD of membrane staining plus the mean OD of cytoplasm staining. In step 46, each cancer cell is identified as being either (i) normalized membrane positive if the normalized membrane optical density of the cancer cell is equal to or less than a normalized membrane threshold, or (ii) normalized membrane negative if the normalized membrane optical density of the cancer cell is greater than the normalized membrane threshold. In step 47, the tissue sample is identified as normalized membrane positive if the percentage of cancer cells that are normalized positive is equal to or greater than a normalized percentage threshold and normalized membrane negative if the percentage of cancer cells that are normalized positive is less than the normalized percentage threshold.

In step 48, each cancer cell is identified as being either optical-density positive if the mean OD of membrane staining is greater than or equal to an optical density threshold, or optical-density negative if the mean OD of membrane staining is less than the optical density threshold. In step 49, the tissue sample is identified as proximity positive if the total percentage of cancer cells that are either optical-density positive or optical-density negative but within a predefined distance of an optical-density positive cancer cell exceeds a proximity percentage threshold. In step 50, the cancer patient is identified as one who will likely benefit from administration of the anti-TROP2 ADC if the tissue sample is both normalized membrane positive and proximity positive.

The optimum parameters for the QCS score were determined by performing a progression free survival (PFS) analysis on clinical trial data. The normalized membrane threshold, the normalized percentage threshold, the optical density threshold and the proximity percentage threshold were correlated to the clinical responses of the cohort of 115 training patients who have been treated with the TROP2 ADC. Thus, the cohort of 115 NSCLC patients was used to validate the predicted response score from the combined QCS features memb(meanOD)/(memb(meanOD)+cyto(meanOD))_q5 and bystander_memb(meanOD)_binary_r50_cut25.

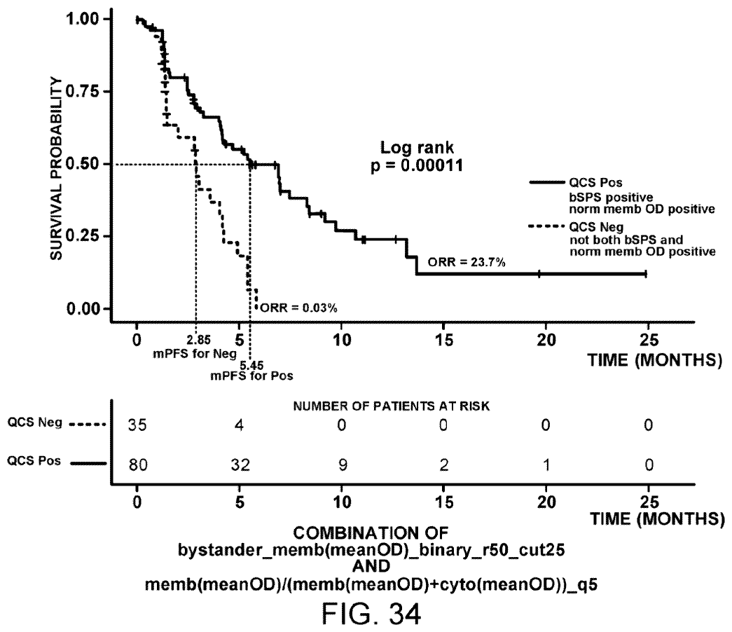

FIG. 34 is a Kaplan-Meier graph of progression-free survival that divides the 115 NSCLC patients of the clinical trial into 80 QCS Positive and 35 QCS Negative patients using method 41 of FIG. 33. The quality of the stratification between the two Kaplan-Meier curves that is achieved using a combination of the bSPS and normalized membrane OD is indicated by the log-rank p value of 0.00011. The group of 80 QCS Positive patients exhibited an objective response rate (ORR) of 23.7% and a mean progression-free survival (mPFS) period of 5.45 months. The group of 35 QCS Negative patients exhibited an objective response rate of 0.03% and a mean progression-free survival period of 2.85 months. The upper Kaplan-Meier curve shows the group of 80 patients with better survival outcomes, which corresponds to patients for which both (i) at least 5% of their cancer cells exhibited a normalized membrane optical density equal to or less than 0.4864, and (ii) at least 92% of the cells either exhibited an optical density of membrane staining of at least 25, or were located within 50 microns of a cell exhibiting the minimal membrane staining of 25. Thus, the scoring method 41 of FIG. 33 identified 80 of the 151 J101 lung cancer patients as likely to benefit from an anti-TROP2 ADC therapy.

The scoring method 41 of FIG. 33 identified only 80 patients for the ADC therapy, whereas the bSPS scoring method 10 of FIG. 13 identified 88 of the 151 J101 patients as likely to benefit from the ADC therapy. This shows that the combined bSPS and normalized membrane OD QCS feature is more selective than the individual features and identifies patients with a higher probability of benefiting from the ADC therapy. Note that the a proximity percentage threshold of at least 92% used in the combined QCS feature is more inclusive than the predetermined threshold of at least 99.97% used in the individual normalized membrane OD feature, and nevertheless 35 QCS Negative patients were excluded from ADC therapy by the combined QCS feature compared to only 27 QCS Negative patients excluded from ADC therapy by the individual binary spatial proximity feature. Thus, a higher proportion of patients are identified as likely to benefit from the ADC based on each individual QCS feature than based on the combined feature of bSPS and normalized membrane OD.

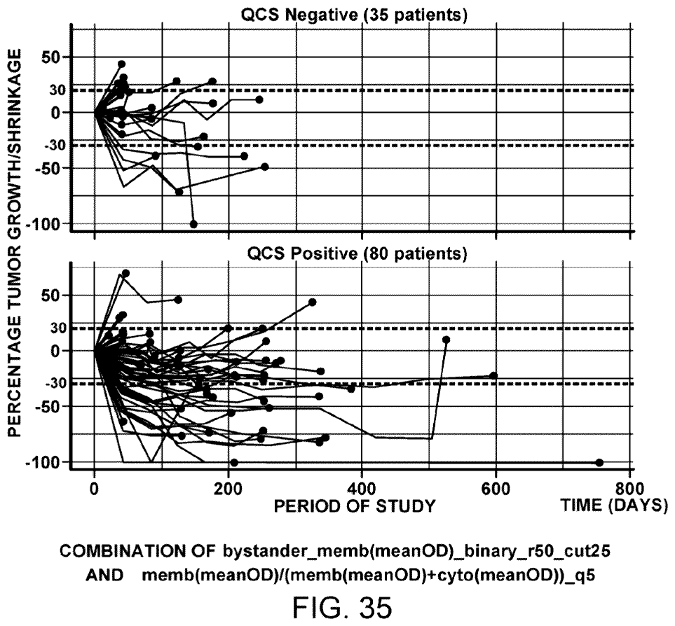

FIG. 35 shows spider plots of the 80 QCS Positive patients and the 35 QCS Negative patients of the Kaplan-Meier curves of FIG. 34. Each dot of the spider plots indicates the percentage by which the patient's tumor grew or shrank after the indicated number of days. The spider plots show that significantly more dots in the lower plot for QCS Positive are below the line for 0% tumor growth, whereas the distribution of dots in the upper plot for QCS Negative patients is weighted above the 0% line.

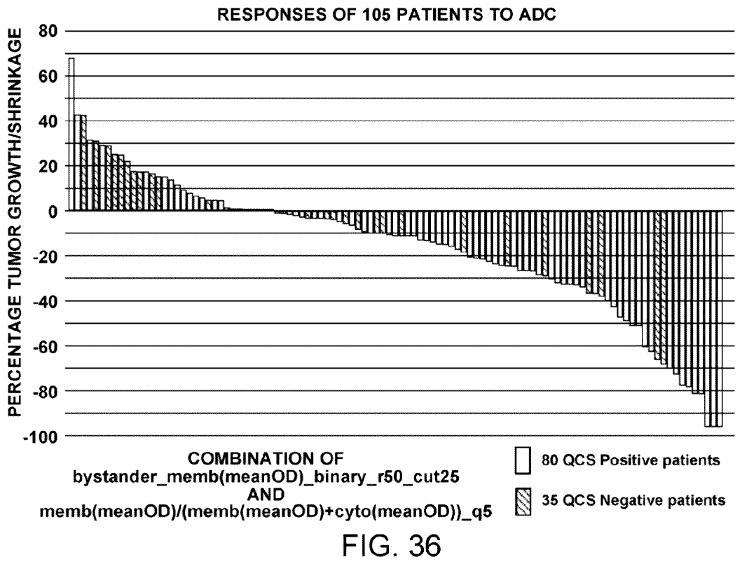

FIG. 36 is a waterfall bar graph showing the clinical response of 105 of the 115 NSCLC patients in the J101 trial ordered from greatest tumor growth to greatest tumor shrinkage. Tumor growth/shrinkage results are not shown for 10 of the non-evaluable (NE) patients. Solid bars denote QCS Positive patients whose stained tissue samples were both normalized membrane positive with the normalized membrane OD score and proximity positive with the binary spatial proximity score. Bars with diagonal hatching denote QCS Negative patients whose stained tissue samples were not both normalized membrane positive and proximity positive. Most of the bars above the line for 0% tumor growth have diagonal hatching denoting QCS Negative patients, while most of the bars below the line for 0% tumor growth are solid bars denoting QCS Positive patients. Thus, the bar graph shows that the combined bSPS and normalized membrane OD QCS feature identifies a greater portion of the patients whose tumors shrank after ADC treatment as QCS Positive and a greater portion of the patients whose tumors grew after ADC treatment as QCS Negative.

Although the present invention has been described in connection with certain specific embodiments for instructional purposes, the present invention is not limited thereto. Various modifications, adaptations, and combinations of various features of the described embodiments can be practiced without departing from the scope of the invention as set forth in the claims.

1-29. (canceled)

30. A method of treating cancer comprising administering to a cancer patient an antibody drug conjugate (ADC) comprising an ADC payload and an ADC antibody that targets a protein on a cancer cell, wherein the protein is calcium signal transducer trophoblast antigen 2 (TROP2), the method comprising:
staining a tissue sample from the cancer patient immunohistochemically using a dye linked to a diagnostic antibody, wherein the diagnostic antibody binds to the protein on the cancer cells in the tissue sample;
acquiring a digital image of the tissue sample;
detecting cancer cells in the digital image;
determining for each cancer cell a mean optical density of staining by the dye in the membrane of the cancer cell;
determining for each cancer cell a mean optical density of staining by the dye in the cytoplasm of the cancer cell;
computing a normalized membrane optical density (nmOD) for each cancer cell in the digital image;
identifying each cancer cell as nmOD positive if the nmOD of the cancer cell is equal to or less than a nmOD threshold; and
identifying the tissue sample as nmOD positive if the percentage of cancer cells that are nmOD positive is equal to or greater than a nmOD percentage threshold;
wherein a therapy comprising the ADC is administered to the cancer patient if the tissue sample is nmOD positive.

31. (canceled)

32. The method of claim 30, wherein the detecting of cancer cells comprises detecting for each cancer cell the pixels that belong to the membrane and the pixels that belong to the cytoplasm.

33. (canceled)

34. The method of claim 30, wherein the nmOD threshold is in a range of 0.45 to 0.50.

35. The method of claim 30, wherein the nmOD threshold is 0.486.

36. The method of claim 30, wherein the nmOD percentage threshold is in a range of 3% to 7%.

37. The method of claim 30, wherein the nmOD percentage threshold is 5%.

38. The method of claim 30, wherein the detecting of cancer cells comprises detecting for each cancer cell the pixels that belong to the membrane using a cell center determined for each cancer cell.

39. The method of claim 30, wherein the staining intensity of each membrane is computed based on an average optical density of a brown diaminobenzidine (DAB) signal in pixels of the membrane, and wherein the staining intensity of each cytoplasm is computed based on the average optical density of the brown DAB signal in pixels of the cytoplasm.

40. The method of claim 30, wherein the dye is 3,3′-Diaminobenzidine (DAB).

41. The method of claim 30, wherein the ADC is datopotamab deruxtecan (DS-1062).

42. The method of claim 30, wherein the ADC antibody is hTINA1-H1L1, or wherein the ADC antibody is a humanized IgG1 monoclonal antibody.

43. (canceled)

44. The method of claim 30, wherein the ADC payload is a topoisomerase I inhibitor.

45. The method of claim 44, wherein the topoisomerase I inhibitor, when released from the antibody-drug conjugate, is represented by the following formula:

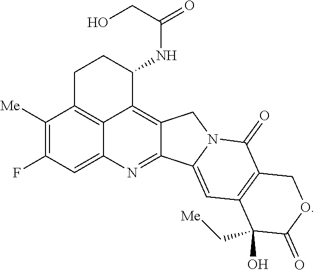 [SMILES: CC[C@@]1(O)C(=O)OCc2c1cc1n(c2=O)Cc2c-1nc1cc(F)c(C)c3c1c2[C@@H](NC(=O)CO)CC3]

46. The method of claim 30, wherein the diagnostic antibody is Abcam EPR 20043.

47. The method of claim 30, wherein the cancer patient has a cancer selected from the group consisting of: breast cancer, gastric cancer, colorectal cancer, lung cancer, esophageal cancer, head-and-neck cancer, esophagogastric junction cancer, biliary tract cancer, Paget's disease, pancreatic cancer, ovarian cancer, uterine cancer sarcoma, bladder cancer, prostate cancer, urothelial cancer, gastrointestinal stromal tumor, uterine cervix cancer, squamous cell carcinoma, peritoneal cancer, liver cancer, hepatocellular cancer, endometrial cancer, kidney cancer, vulval cancer, thyroid cancer, penis cancer, leukemia, malignant lymphoma, plasmacytoma, myeloma, glioblastoma multiforme, sarcoma, osteosarcoma, and melanoma.

48. The method of claim 30, wherein the cancer patient has a cancer selected from the group consisting of: breast cancer, gastric cancer, colorectal cancer, lung cancer, pancreatic cancer, ovarian cancer, prostate cancer, and kidney cancer.

49. The method of claim 30, wherein the cancer patient has breast cancer.

50. The method of claim 30, wherein the cancer patient has lung cancer.

51. The method of claim 30, wherein the cancer patient has non-small cell lung cancer.

52. The method of claim 30, wherein the ADC comprises an anti-TROP2 antibody conjugated to a drug-linker via a thioether bond, wherein the drug-linker is represented by the following formula: 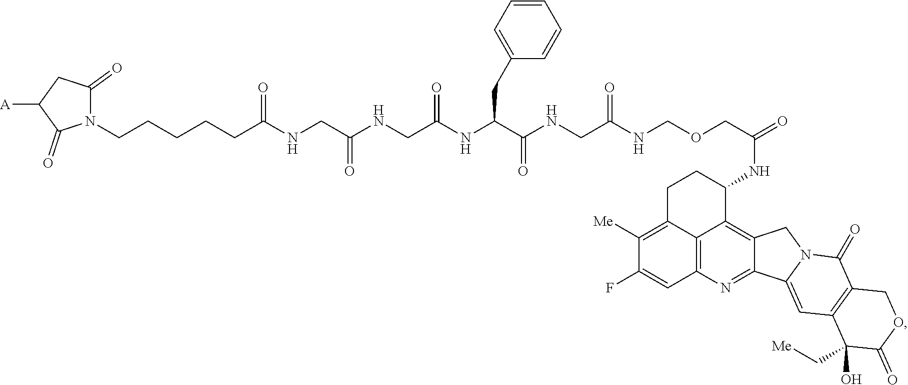
and wherein A represents a connecting position to the anti-TROP2 antibody.

53. The method of claim 30, wherein the ADC comprises includes an anti-TROP2 antibody comprising:
(i) a heavy chain comprising a CDRH1 consisting of an amino acid sequence represented by SEQ ID NO: 3, a CDRH2 consisting of an amino acid sequence represented by SEQ ID NO: 4, and a CDRH3 consisting of an amino acid sequence represented by SEQ ID NO: 5; and
a light chain comprising a CDRL1 consisting of an amino acid sequence represented by SEQ ID NO: 6, a CDRL2 consisting of an amino acid sequence represented by SEQ ID NO: 7, and a CDRL3 consisting of an amino acid sequence represented by SEQ ID NO: 8; or
(ii) a heavy chain variable region consisting of the amino acid sequence represented by SEQ ID NO: 9; and
a light chain variable region consisting of the amino acid sequence represented by SEQ ID NO: 10; or
(iii) a heavy chain consisting of the amino acid sequence represented by SEQ ID NO: 11; and
a light chain consisting of the amino acid sequence represented by SEQ ID NO: 2.

54-55. (canceled)

56. The method of claim 41, wherein the ADC comprises an anti-TROP2 antibody comprising:
a heavy chain consisting of the amino acid sequence represented by SEQ ID NO: 1; and
a light chain consisting of the amino acid sequence represented by SEQ ID NO: 2.

57-114. (canceled)
